(19) United States
(12) Patent Application Publication
(10) Pub. No.: US 2007/0134794 A1
(43) Pub. Date: Jun. 14, 2007

(54) Strategies for the identification and isolation of cancer stem cells and non-cancerous stem cells

(75) Inventor: Joseph A. Mangano, Arlington, VA (US)

Correspondence Address: WOLF GREENFIELD & SACKS, P.C., 600 ATLANTIC AVENUE, BOSTON, MA, 02210-2206, US

(73) Assignee: Science Research Laboraory, Inc., Somerville, MA (US)

(21) Appl. No.: 11/704,004

(22) Filed: Feb. 8, 2007

Related U.S. Application Data
(63) Continuation of application No. 10/272,536, filed on Oct. 15, 2002, now abandoned.
(60) Provisional application No. 60/329,285, filed on Oct. 15, 2001.

(51) Int. Cl. C12N 5/08; C12N 5/06

(52) U.S. Cl. 435/325; 435/366

(57) ABSTRACT

The present invention, in some embodiments, describes methods for selecting and enriching specific types of stem cells and/or progenitor cells from a sample containing at least two different types of stem cells and/or progenitor cells. In one embodiment, the method involves selecting and enriching cancer stem cells and/or cancer progenitor cells. In other embodiments, the invention involves improved methods for purging cancer cells from autologous or allogenic transplants prior to reinjection into a patient. In other embodiments, the invention describes improved methods to screen for the efficacy of drug candidate for affecting the function and/or viability of a specific type of stem cell or progenitor cell, for example a cancer stem cell. In this context, the inventive method can involve screening the effectiveness of chemotherapeutic agents in completely eliminating all cancer cells (i.e. mature cancer cells and cancer stem/progenitor cells). In yet other embodiments, the invention provides methods for the identification of a particular type or sub-population of stem cells or progenitor cells from a mixed cell-type population. In one embodiment of such a method, the invention describes a method for the early detection of metastatic cancer from simple tests on blood or bone marrow. Stem cell culture methods are also disclosed which utilize cell lysates to preferentially induce either asymmetric or symmetric division of the stem cells. The invention also provides, in some embodiments, novel stem cell suspensions that include, in some instances enriched suspensions of particular types or sub-populations of stem cells or progenitor cells, for example cancer stem cells. In other embodiments, the invention provides a suspension of stem cells that is substantially free of all stem cells of a particular type or sub-population and, optionally, also substantially free of all mature cells. In one example of such an embodiment, the invention provides a suspension of stem cells that is substantially free of all cancer cells (i.e. mature cancer cells as well as cancer stem cells and cancer progenitor cells).

### RELATED APPLICATIONS

This application is a continuation of U.S. patent application Ser. No. 10/272,536, filed Oct. 15, 2002, which claims priority to U.S. Provisional Patent Application Ser. No. 60/329,285, filed Oct. 15, 2001, both of which are incorporated herein by reference.

### BACKGROUND

The identification and/or isolation of cancer and non-cancerous stem cells, as provided by the current invention, can be useful for enabling a wide variety of medical diagnostic methods or treatment methods, as well as for enabling drug screening assays directed to developing or identifying drugs effective against cancer regenerating cells (i.e., cancer stem cells and/or progenitor cells). For example, identification and/or isolation of cancer stem cells and non-cancerous stem cells according to the teachings of the current invention can enable a variety of new treatments, diagnostic protocols, and drug screening assays for many forms of cancer including breast cancer, ovarian cancer, lymphomas, leukemias, and myelomas. Currently, treatment of these cancers is benefiting from the development of high dose chemotherapy or radiation followed by autologous or allogenic bone marrow or stem cell transplant. In this strategy, bone marrow or mobilized peripheral blood is harvested before high dose chemotherapy or radiation and after any surgery to remove as much of a malignant tumor as possible from a patient. High doses of chemotherapeutic agents and/or radiation are then administered, with patient recovery made possible by subsequent injection of the bone marrow or stem cells to reconstitute the patient's immune system, which was ablated by the intense doses of chemotherapy and/or radiation. This therapy has seen some success in the treatment of a variety of cancers, for example breast cancer and hematopoietic cancers, at cancer centers across the United States.

At least two issues remain unresolved, however. First, results from clinical trials with such currently available treatments, though sometimes statistically favorable, are not consistent from patient to patient. Secondly, for many late stage cancers, the overall survival statistics are often quite poor. Therefore, improvements in high dose chemotherapy are urgently needed, especially for advanced, and/or metastatic cancers.

High dose chemotherapy followed by autologous or allogenic transplant is limited by at least two major factors: (1) existing chemotherapeutic agents are not as effective as required against late stage/metastatic cancers, and (2) clinical methods of purging cancer cells from the transplants prior to injection are not as effective as desirable at eliminating all residual cancer forming cells. Thus, there exists a need for improved chemotherapeutic agents and improved methods to screen the efficacy of such agents in completely eliminating cancer cells. There also exists a need for improved methods for removing cancer cells from transplant grafts prior to reinjection into a patient. In addition, there exists a need to provide early detection of cancer cells from simple tests on blood or marrow samples.

The existence of primitive cancerous stem cells for hematopoietic cancers and several types of solid tumors has been demonstrated (see e.g., Cooper, G. M. Elements of Human Cancer, Jones and Bartlett Publishers, 1992, ISBN: 0867201916 (hereinafter “Cooper”); Bonnet D. and Dick J. E. “Human acute myeloid leukemia is organized as a hierarchy that originates from a primitive hematopoietic cell,” Nature Medicine, 3: pp. 730-737 (1997) (hereinafter “Bonnet and Dick”); Park C. H., Bergsagel D. E., and McCulloch E. A. “Mouse myeloma tumor stem cells: a primary cell culture assay,” J. Nat. Cancer Inst., 46: pp. 411-422 (1971) (hereinafter “Park et al.”); Hamburger A. W. and Salmon S. E. “Primary bioassay of human tumor stem cells,” Science, 197: pp. 461-463 (1977) (hereinafter “Salmon I”); and U.S. Pat. No. 4,411,990 to Salmon, et al. entitled “Primary bioassay of human tumor stem cells, issued Oct. 25, 1983 (hereinafter “Salmon II”)). Typical current methods for diagnosing or treating cancer, removing cancer cells from transplant grafts prior to injection into a patient, or methods to screen the efficacy of anti-cancer agents in completely eliminating cancer cells, do not account for the presence of cancer stem cells, which can propagate, differentiate into mature cancer cells and self-renew, thereby reforming cancers and leading to remissions. Accordingly, there exists a need for new methods for diagnosing or treating cancer, removing cancer cells from transplant grafts prior to injection into a patient, and methods to screen the efficacy of anti-cancer agents in eliminating cancer cells, which account for and/or are specifically directed to cancer stem cells and/or progenitor cells.

### SUMMARY OF THE INVENTION

The present invention, in some embodiments, describes methods for selecting and enriching specific types of stem cells and/or progenitor cells from a sample containing at least two different types of stem cells and/or progenitor cells. In one exemplary embodiment, the method involves selecting and enriching cancer stem cells and/or cancer progenitor cells. In other embodiments, the invention involves improved methods for purging cancer cells from autologous or allogenic transplants prior to injection into a patient. In other embodiments, the invention describes improved methods to screen for the efficacy of drug candidate for affecting the function and/or viability of a specific type of stem cell or progenitor cell, for example a cancer stem cell. In this context, the inventive method can involve screening the effectiveness of chemotherapeutic agents in eliminating substantially all cancer cells (i.e. mature cancer cells and cancer stem/progenitor cells). In yet other embodiments, the invention provides methods for the identification of a particular type or sub-population of stem cells or progenitor cells from a mixed cell-type population. In one embodiment of such a method, the invention describes a method for the early detection of metastatic cancer from simple tests on blood or bone marrow. The invention also can provide, in some embodiments, novel stem cell suspensions that include, in some instances substantially pure suspensions of particular types or sub-populations of stem cells or progenitor cells, for example cancer stem cells. In other embodiments, the invention can provide a suspension of stem cells that is substantially free of all stem cells of a particular type or sub-population and, optionally, also substantially free of all mature cells. In one example of such an embodiment, the invention can provide a suspension of stem cells that is substantially free of all cancer cells (i.e. mature cancer cells as well as cancer stem cells and cancer progenitor cells).

In some embodiments, the invention involves methods for propagating stem cells under culture conditions selected to favor either symmetric division of the stem cells, whereby each dividing stem cell produces two identical daughter stem cells, or asymmetric division, whereby each dividing stem cells produces one identical daughter stem cell and one daughter cell that is more differentiated than the stem cell. In certain embodiments of such methods, lysates produced from cells of at least one selected differentiated cell type are added to the stem cell cultures so as to induce the stem cells to propagate, depending on the concentration of the lysates, to undergo either symmetric mitosis or asymmetric mitosis. Such methods can also be utilized to favor stem cell differentiation along certain lines to produce particularly desirable mature stem cell types in culture.

In one aspect, the invention can provide a suspension of human cells including cancer stem cells and/or cancer progenitor cells. The suspension is substantially free of mature cancer cells and lympho-hematopoietic cells.

In another embodiment, the invention can provide a suspension of human cells including cancer stem cells and/or cancer progenitor cells, and lympho-hematopoietic stem cells and/or lympho-hematopoietic progenitor cells. The suspension is substantially free of mature cancer cells and mature lympho-hematopoietic cells.

In yet another embodiment, the invention can provide a suspension of human cells including cancer stem cells and/or cancer progenitor cells, and pluripotent lympho-hematopoietic cells. The suspension is substantially free of mature cancer cells and mature lympho-hematopoietic cells.

In another embodiment, the invention can provide a suspension of pluripotent or multipotent lympho-hematopoietic stem cells and/or lympho-hematopoietic progenitor cells that is substantially free of all cancer cells including, but not limited to, cancer stem cells, cancer progenitor cells, and mature cancer cells. The suspension is also substantially free of mature lympho-hematopoietic cells.

In another aspect, the invention can provide a cell suspension that includes cancer stem cells and/or cancer progenitor cells. The suspension is substantially free of mature cancer cells and mature non-cancer cells.

In another embodiment, the invention can provide a cell suspension including stem cells and/or progenitor cells. The suspension is substantially free of mature cancer cells, cancer stem cells and cancer progenitor cells, and mature non-cancerous cells. The cell suspension is derived from an initial cell suspension that includes mature cancer cells, and cancer stem cells and/or cancer progenitor cells.

In one aspect, a method for creating from a suspension of biological cells having a given cell population of at least a first and a second stem and/or progenitor cell type and including mature cells, a selected suspension containing a selected viable subpopulation of the given cell population, with the selected subpopulation including cells of the first stem and/or progenitor cell type and being substantially free of mature cells and cells of the second stem and/or progenitor cell type is disclosed. The method involves selecting stem cells and/or progenitor cells to form a stem cell and/or progenitor cell suspension comprising cells of at least the first and the second stem and/or progenitor cell type, and selecting stem cells and/or progenitor cells of the first type from the stem cell and/or progenitor cell suspension created in the above step.

In another aspect, a method for creating from a suspension of biological cells having a given cell population of at least a first and a second stem and/or progenitor cell type and including mature cells, a selected suspension containing a selected viable subpopulation of the given cell population, with the selected subpopulation including cells of the first stem and/or progenitor cell type and being substantially free of mature cells and cells of the second stem and/or progenitor cell type is disclosed. The method involves selecting stem cells and/or progenitor cells of the first type from said given cell population.

In yet another aspect, a method for creating from a suspension of biological cells having a given cell population of at least a first and a second stem and/or progenitor cell type and including mature cells, a selected suspension containing a selected viable subpopulation of the given cell population, with the selected subpopulation including cells of the first stem and/or progenitor cell type and being substantially free of cells of the second stem and/or progenitor cell type is disclosed. The method involves selecting stem cells and/or progenitor cells to form a stem cell and/or progenitor cell suspension comprising cells of at least the first and the second stem and/or progenitor cell type, dividing the stem cell and/or progenitor cell suspension into a plurality of colony-forming units, each containing at least one viable cell, spatially separating the colony-forming units, expanding the colony-forming units so that the viable cells in the colony-forming units differentiate and increase in concentration, performing analytical tests on the cells of at least one of the colony-forming units to detect the presence of mature cells of a lineage derived from the first and the second cell types, retaining cells of at least one colony-forming unit including mature cells of a lineage derived from the first cell type but not including mature cells of a lineage derived from the second cell type as determined by the analytical tests performed in the previous step.

In another aspect the invention provides a method for determining if a cancer is metastatic, for use in selecting a method of cancer treatment, by detecting the presence of and type of cancer stem cells and/or cancer progenitor cells found in peripheral blood or bone marrow samples.

In yet another aspect, the invention provides a method for classifying a metastatic cancer for use in selecting a method of cancer treatment by detecting and determining the type of, and relative abundance of, cancer stem cells and/or cancer progenitor cells found in peripheral blood or bone marrow samples.

In another aspect, the invention provides a method of enriching and then measuring the number and type of cancer stem cells and/or cancer progenitor cells in peripheral blood samples for detecting and classifying metastatic cancer, including micro-metastases, at an early stage.

In another aspect, the invention provides a method of enriching and then measuring the number and type of cancer stem cells and/or cancer progenitor cells in bone marrow samples for detecting and classifying metastatic cancer, including micro-metastases, at an early stage.

In yet another aspect, the invention provides an in vitro method of screening and assessing efficacy of new and existing therapeutic agents in killing cancer stem cells and/or cancer progenitor cells.

In another aspect, the invention provides a method for screening chemotherapeutic agents, or other cancer treatment protocols, for efficacy against cancer stem cells and/or cancer progenitor cells. The method involves forming from a first suspension of cancer cells that includes mature cancer cells, and cancer stem cells and/or cancer progenitor cells, a second suspension of viable cells that includes cancer stem cells and/or cancer progenitor cells and is substantially free of mature cancer cells. The second suspension is then treated with the chemotherapeutic agent to be tested and subsequently cultured and expanded. At least one analytical test is then performed on the second suspension to detect the presence of viable, mature cancer cells.

In yet another aspect, the invention provides a method for screening chemotherapeutic agents, or other cancer treatment protocols, for efficacy against cancer stem cells and/or cancer progenitor cells and for toxicity against non-cancerous stem cells and/or non-cancerous progenitor cells. The invention involves forming from a first suspension of cells that includes mature cancer cells, mature non-cancerous cells, cancer stem cells and/or cancer progenitor cells, and non-cancerous stem cells and/or non-cancerous progenitor cells, a second suspension of viable cells that includes cancer stem cells and/or cancer progenitor cells, and non-cancerous stem cells and/or non-cancerous progenitor cells and is substantially free of any mature cells. The second suspension is treated with a chemotherapeutic agent, or other cancer treatment protocols, to be tested and subsequently cultured and expanded. At least one analytical test is then performed on the second suspension to detect the presence of viable, mature cancer cells and viable non-cancerous cells.

In another aspect, a method for obtaining a desired type of stem and/or progenitor cell from a mixture of cell types is disclosed. The method comprises forming a treated suspension by enriching an initial suspension of biological cells, having a given population including mature cancer cells, mature non-cancerous cells, cancer stem cells and/or progenitor cells, and non-cancerous stem cells and/or progenitor cells, in the cancer stem cells and/or progenitor cells and the non-cancerous stem cells and/or progenitor cells by inactivating and/or removing from the precursor suspension a substantial fraction of the mature cells of each cell type contained in the precursor suspension. The method further involves separating the cancer stem cells and/or progenitor cells from the non-cancerous stem cells and/or progenitor cells.

In yet another aspect, a method for reconstituting the hematopoietic system of a human patient in need thereof is disclosed. The method comprises obtaining a suspension of bone marrow derived cells and/or peripheral blood derived cells, the suspension having a given cell population including cancer stem cells and/or progenitor cells and non-cancerous stem cells and/or progenitor cells. The method further involves purifying the non-cancerous stem cells and/or progenitor cells from the cancer stem cells and/or progenitor cells to form a suspension of non-cancerous stem cells and/or progenitor cells substantially free of any cancer cells, and engrafting the patient with this suspension and/or cells derived therefrom.

In another aspect, a method for propagating stem cells is disclosed. The method involves exposing stem cells in culture to a concentration of lysate produced from cells of at least one selected differentiated cell type, the concentration able to induce the stem cells to propagate by preferentially undergoing either symmetric mitosis, whereby each dividing stem cell produces two identical daughter stem cells, or asymmetric mitosis, whereby each dividing stem cell produces one identical daughter stem cell and one daughter cell that is more differentiated than the stem cells.

In yet another aspect, a method for obtaining a desired type of stem and/or progenitor cell for a mixture of cell types is disclosed. The method comprises subjecting an initial suspension of biological cells, having a given cell population including mature cancer cells, mature non-cancerous cells, cancer stem cells and/or progenitor cells, and non-cancerous stem cells and/or progenitor cells, to electric field conditions sufficient to porate a substantial fraction of cells that are not stem cells and/or progenitor cells while maintaining substantially viable the stem cells and/or progenitor cells in the precursor suspension. The method further involves selectively inactivating a substantial fraction of the porated cells that are not stem cells and/or progenitor cells while maintaining substantially viable the stem cells and/or progenitor cells in the suspension subjected to the electric field. The cancer stem cells and/or progenitor cells are then separated from the non-cancerous stem cells and/or progenitor cells.

In yet another aspect, a method for obtaining a desired type of stem cell from a mixture of cell types is disclosed. The method comprises forming a treated cell suspension by enriching an initial suspension of biological cells, having a given cell population including mature cancer cells, mature non-cancerous cells, cancer stem cells, and non-cancerous stem cells, in the cancer stem cells and the non-cancerous stem cells by inactivating and/or removing from the initial suspension a substantial fraction of the mature cells of each cell type contained in the initial suspension. The method also involves spatially segregating the cells of the treated cell suspension into a plurality of colony-forming units and expanding the colony-forming units so that the viable stem cells in the colony-forming units differentiate and increase in number and exposing the colony-forming units to a concentration of lysate produced from cells of at least one selected differentiated cell type, the concentration of lysate being able to induce the stem cells to propagate by preferentially undergoing asymmetric mitosis, whereby each dividing stem cell produces one identical daughter stem cell and one daughter cell that is more differentiated than the stem cells. The method further involves performing at least one analytical test on at least one of the expanded colony-forming units to detect the presence of mature cancer stem cells and/or mature non-cancerous cells, and retaining the cells of at least one of the expanded colony-forming units having a desired cell type as determined by at least one analytical test, as performed above.

In yet another aspect, a method for detecting and classifying metatastic cancer in a tissue or blood sample of a patient, which includes a suspension of cells, is disclosed. The method involves selecting stem cells and/or progenitor cells from the suspension of cells to form a second suspension enriched in stem cells and/or progenitor cells and substantially free of mature cells. The method further involves expanding the second suspension so that the viable stem cells and/or progenitor cells differentiate and increase in number, thereby producing an expanded suspension. The method involves performing at least one analytical test on the expanded suspension to detect the presence of mature cancer cells, and estimating a relative abundance of cancer stem cells and/or progenitor cells present in the suspension of cells included in the tissue sample.

Other advantages, novel features, and uses of the invention will become more apparent from the following detailed description of non-limiting embodiments of the invention when considered in conjunction with the accompanying drawings, which are schematic and which are not intended to be drawn to scale. In the figures, each identical, or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention. In cases where the present specification and a document incorporated by reference include conflicting disclosure, the present specification shall control.

### BRIEF DESCRIPTION OF THE DRAWINGS

FIG. 1a is a flow diagram outlining the steps in a method for cancer stem cell and/or non-cancerous stem cell isolation according to one embodiment of the invention;

FIG. 1b is a flow diagram outlining methods for further treatment, expansion and use of the cell suspensions produced according to the method outlined in FIG. 1a;

FIG. 2 is a flow diagram illustrating stem cell propagation by symmetric division/mitosis (left) and asymmetric division/mitosis (right);

FIG. 3 is a flow diagram outlining the steps in a method for propagating stem cells with various dilutions of cell lysate to determine appropriate concentrations of lysate for favoring symmetric and asymmetric division of the stem cells according to one embodiment of the invention;

FIG. 4 is a flow diagram outlining the steps in a method for screening chemotherapeutic agents and cancer treatment protocols according to one embodiment of the invention.

### DETAILED DESCRIPTION OF THE INVENTION

The present invention provides novel and improved methods for purging cancer cells from transplant tissue containing stem cells, including cancer stem cells and, more generally, for isolating a desired type(s) of stem and/or progenitor cells from a mixture of stem/progenitor cell types. The invention also involves novel methods for propagating stem cells to preferentially undergo either symmetric mitosis, whereby each dividing stem cell produces two identical daughter stem cells, or asymmetric mitosis, whereby each stem cell produces one identical daughter stem cell and one daughter stem cell that is more differentiated than the stem cells, which methods involve exposing stem cells in culture to concentrations of lysate produced from cells of at least one selected differentiated cell type. The invention also provides novel techniques for screening drug candidates for efficacy against, toxicity towards, or effect on particular type(s) or sub-populations of stem cells or progenitor cells (e.g., cancer stem cells/progenitor cells), as well as methods for detecting and characterizing the presence of particular type(s) or sub-populations of stem cells or progenitor cells in a sample. In the context of methods of cancer treatment and diagnostics, the invention provides novel techniques for screening chemotherapeutic agents for efficacy, and novel methods for detecting and characterizing the presence of metastases, especially micro-metastases, of cancer in a patient. It should be noted that, in many of the embodiments discussed below, cancer stem cells/progenitor cells are the specific stem/progenitor cell type(s) being detected, isolated, purged, etc. Such examples are highlighted herein because of their profound importance in the treatment, detection, and study of human cancer; however, it should be kept in mind that essentially any of the below-described methods could also be applied to the detection, isolation, purging, etc., of essentially any known type(s) or sub-population(s) of stem cell and/or progenitor cell from a population including a plurality of types/sub-populations of stem/progenitor cells within the scope of the invention.

“Micro-metastases” as used herein refers to cancer cells derived from a primary malignancy that are freely circulating in the blood or present in the bone marrow. A “macro metastasis” as used herein refers to an established secondary malignancy derived, but separate from the primary malignancy. The methods of the present invention are typically utilized with samples of biological cells, most typically human cells, in suspension that include stem cells and/or progenitor cells. The cell suspensions also, for some embodiments, will contain cancer cells. “Stem cell” as used herein, refers to an essentially indefinitely self-replicating cell of the body that can divide (see discussions of asymmetric versus symmetric mitosis below) to produce one cell like itself and another cell that will follow one or more particular developmental pathways and that is a developmentally primitive and an early progenitor of the more differentiated cells derived therefrom. Stem cells can also be defined functionally as those cells that are capable of long-term clonogenicity (or, in the case of solid tumor stem cells, tumorigenicity) when engrafted into an immunodeficient animal in a xenograft assay For example, lympho-hematopoietic stem cells, which are present in the bone marrow and peripheral blood, give rise to all of the mature cells that characterize the blood, such as red cells, platelets, mononuclear leukocytes, lymphocytes, polymorphonuclear leukocytes, granulocytes, etc.

“Progenitor cells” as used herein, refers to cells that are not fully mature or differentiated, but are less developmentally primitive than stem cells and which are progeny of a less differentiated stem cell. In contrast to stem cells, progenitor cells are not believed to be typically indefinitely self-replicating, and, functionally, are not typically capable of long-term clonogenicity in immunodeficient animals in xenograft assays.

Stem cells and/or progenitor cells can be pluripotent or multipotent(i.e. giving rise to a plurality or multiplicity of mature cell types), or unipotent (i.e. committed to a particular lineage path yielding a single type of mature cell). In the description of the inventive methods to follow, it should be understood that the inventive techniques may be employed with and/or be potentially configured and carried out so as to yield samples containing either or both stem and progenitor cells. Where either term is used separately herein, it is to be understood that unless specifically stated otherwise, both stem cells and progenitor cells may potentially be used or derived and that samples or solutions of “stem cells” may potentially also include progenitor cells. “Fully mature cells” as used herein refers to differentiated cells that represent a final or near final stage of development for a given cell lineage. “Differentiated cells” as used herein refers to cells of a particular lineage that are more mature than stem cells and progenitor cells but are not necessarily at their final developmental stage. The term “mature cells” as used herein encompasses fully mature (i.e. fully developed and terminally differentiated) cells and can also include, in some embodiments, differentiated cells that are not fully mature. Mature cells are not typically capable of self-replication.

The stem cell containing suspensions useful in the context of the present invention can be derived from a variety of sources including, but not limited to, bone marrow, mobilized or unmobilized peripheral blood, umbilical cord blood, fetal liver tissue, other organ tissue, skin, nerve tissue, solid tumors, etc. A variety of stem/progenitor cell types may advantageously be isolated and enriched according to the invention including, but not limited to, lympho-hematopoietic stem cells, multipotent or pluripotent stem cells, mesenchymal stem cells, epithelial stem cells, gut stem cells, skin stem cells, neural stem cells, liver progenitor cells, and endocrine progenitor cells. One embodiment of the invention involves the isolation and purification of lympho-hematopoietic stem cells, which are capable of differentiating into members of the lymphoid, erythroid, and myeloid lineages, from cell suspensions including mature and lineage committed cells to provide a suspension of lympho-hematopoietic stem cells that is substantially free of mature cells, and, in some embodiments, substantially free of cancer cells, including cancer stem cells. The enriched stem cell suspensions according to the present method can, also be advantageously enriched in pluripotent and/or multipotent stem cells of one or more stem cell types, which have the ability to differentiate into the full complement of mature cells derived from the particular type(s) of stem cells. Also, in some embodiments, the enriched stem cell suspensions produced according to the invention can contain, in addition to pluripotent and/or multipotent, stem cells, progenitor cells which are committed blast forming cells and, in certain embodiments, mature cells of particular desired types. For example, for samples including or consisting of hematopoietic stem cells, the enriched suspensions can advantageously also include viable blast forming cells for granulocytes and macrophages (CFC-GM), colony forming cells for erythrocytes (BFU-E), colony forming cells for eosinophils (CFC-Eo), multi-lineage blast forming cells (CFC-GEMM), and immature lymphoid progenitor cells.

The term “substantially free of”, as used in the context of “substantially free of” all cancer cells, including cancer stem cells, and, more generally, in the context of a cell sample or suspension being “substantially free of” a particular type of cell refers to such sample or suspension having either none of the cells that the sample or suspension is “substantially free of” or having only so many of such cells that the sample or suspension is functionally equivalent to a sample or suspension entirely free of such cells for whatever end use such sample or suspension is applied. A cell sample or suspension that is “substantially free of” cancer cells or “substantially free of” non-cancerous cells, as used herein, can be defined functionally—if, upon culture and expansion of the cell suspension or sample (and/or engraftment of the cell suspension or sample into an immunodeficient animal), no cells derived from cancerous or non-cancerous cells, respectively, can be detected, then such sample or suspension is “substantially free” of such cells.

To understand the new strategies provided by certain cancer-related embodiments of the invention, it is important to understand the characteristics of the cancer stem cell. A cancer stem cell or cancer progenitor cell, as used herein, is believed to be a normal stem cell or progenitor cell, which has been transformed in some manner, for example by mutation due to genetic, environmental, other, or a combination of causes, so that it produces a cellular lineage including mature cancer cells, which are typically associated with the various macroscopically observed tumors and malignancies of the body. In this regard, the cancer stem cell is a primitive progenitor cell of this cancer cell lineage.

For example, transformed lympho-hematopoietic stem cells can form hematopoietic cancer stem cells whose lineages lead to the various forms of leukemias that are macroscopically observed (see Cooper, p. 269, and Bonnet and Dick (both Cooper, and Bonnet and Dick are hereby incorporated herein by reference)). It is believed that lympho-hematopoietic stem cells and/or progenitor cells can be transformed in several different ways, giving rise to the different observed cancers of the blood and immune system. Thus there could be several cancer stem cell types for each non-cancerous stem cell or progenitor cell type.

In another example, certain pathological transformations of epithelial stem cells or epithelial progenitor cells have been recently demonstrated to form epithelial cancer stem cells that can produce various forms of breast and ovarian cancers and other epithelial cancers (See U.S. patent application Ser. No. 09/920,517, to Clarke et al., published as U.S. Patent Application Publication No. 2002/0119565 A1, on Aug. 29, 2002, and International application no. PCT/US01/24243, published as International publication no. WO 02/12447 A2 on Feb. 14, 2002, also to Clarke et al., (hereinafter collectively referred to as “Clarke et al.”), each of which is incorporated herein by reference). Similarly, stem cells associated with other organs and tissue compartments of the body can be transformed to form cancer stem cells that lead to the various other forms of cancer observed macroscopically in the body, for example, skin cancer, lung cancer, digestive system cancers, oral cancer, and prostate cancer. In addition, the cancer stem cell would replicate its own transformed form rather than the non-cancerous version of the stem cell. Each of such cancer stem cells can be separated from non-cancerous stem cells and, optionally, mature cells by the methods provided according to the invention.

“Separating” or “isolating” a cancer stem cell from a non-cancerous stem cell, or, more generally, one cell type from another cell type, as used herein, refers to spatially segregating cells of the different cell types from each other so as to yield a fraction that is relatively enriched in a first cell type, with respect to a second cell type, and another fraction that is relatively enriched in a second cell type, with respect to a first cell type. In certain embodiments, cell types (e.g., cancer stem cells and/or progenitor cells and non-cancerous stem cells and/or progenitor cells) can be separated from each other such that the segregated fractions of the respective cell types are enriched in the desired cells by at least a factor of about 5, in some embodiments by at least a factor of about 10, in some embodiments by at least a factor of about 100, in some embodiments by at least a factor of about 1000, in some embodiments by at least a factor of about 10⁴, in some embodiments by at least a factor of about 10⁵, in some embodiments by at least a factor of about 10⁶, and in yet other embodiments the desired cells in the segregated fraction are free of cells of the undesired type.

It is believed that cancer stem cells can form the foci of observed micro-metastases and macro-metastases in metastatic cancers, as these what are believed to be small, normally quiescent cells migrate away from the original tumor site. Normally quiescent cancer stem cells also would tend to be essentially impervious to conventional chemotherapeutic agents, which are primarily effective against rapidly growing and dividing mature cells. Accordingly, outgrowth of these cancer stem cells may be a primary agent responsible for relapse of a patient after a remission brought about by the beneficial action of conventional chemotherapeutic agents. In addition, at least some cancer stem and/or progenitor cells would be expected to possess cell surface markers typically employed by prior art affinity based stem cell isolation and lack those typically employed by cancer cell purging techniques. Furthermore, it is also believed that at least some of the most primitive and/or pluripotent and/or multipotent stem cells, including certain cancer stem cells, may lack the cell surface markers typically employed by prior art affinity-based stem cell isolation and cancer cell purging techniques. Thus the existence of cancer stem cells necessitates improved strategies in the search for more effective chemotherapeutic agents and cancer therapies, and also necessitates more effective purging of cancer cells from transplants prior to re-injection into a patient to reconstitute the patient's immune and blood system.

In one particular embodiment, the current invention provides methods for separating primitive cancer stem cells and/or cancer progenitor cells from non-cancerous stem cells and/or progenitor cells in samples containing a mixture of immature and mature cell types. The methods of such embodiments of the invention can be advantageously used to produce novel cell suspensions containing, for example, cancer stem cells and/or cancer progenitor cells, which are substantially free of mature cells (including both mature cancer and mature non-cancerous cells), and in some embodiments, are also substantially free of non-cancerous stem cells/progenitor cells. Such suspensions of cells can be advantageously used for performing the novel chemotherapeutic/cancer treatment screening methods, described in more detail herein, to screen for agents that have efficacy in killing cancer stem cells and/or progenitor cells, and also for the novel methods described herein for detecting, classifying, and enumerating human cancers.

The term “suspension of cells” or “cellular suspension” as used herein refers to a mixture of cells suspended in a carrier liquid. The carrier liquid may be naturally part of the biological sample from which the cells derive, for example blood is a suspension of blood cells suspended in plasma, or, for cells which are not normally present in a suspension, the carrier liquid can be any suitable diluent or medium. In another embodiment, the invention provides methods for creating a cellular suspension including a plurality of stem cells of one or more specific and desired types, for example lympho-hematopoietic stem cells, which includes primitive pluripotent and/or multipotent stem cells and is substantially free of all cells not of the desired stem cell type(s), such as mature cells and stem/progenitor cells of type(s) different from the specific and desired type(s), wherein the suspension is created from a precursor cellular suspension containing the stem/progenitor cells different from the specific and desired type(s). For example, for such an embodiment in the context of cancer treatment, diagnostics, or research, the invention provides methods for creating a cellular suspension including a plurality of non-cancerous stem cells, for example lympho-hematopoietic stem cells, which may include primitive pluripotent and/or multipotent stem cells and is substantially free of all cancer cells, including mature cancer cells and cancer stem/progenitor cells, from a precursor cellular suspension containing cancer stem/progenitor cells. Such suspensions can advantageously be used to reconstitute the blood and immune systems of patients in need of a bone marrow transplantation or, equivalently, in need of hematopoietic reconstitution, for example, after immunoablative chemotherapy and/or radiation, with less danger of a cancer relapse due to reinfusion of cancer cells, and especially cancer stem cells.

The current invention, in some embodiments involves methods to allow cancer stem cells to be separated from non-cancerous stem cells, (and, optionally, mature cells), identified and studied. In other embodiments, the inventive methods involve isolation of non-cancerous cells, including stem cells and/or progenitor cells, that are substantially free of all cancer cells, including cancer stem cells and cancer progenitor cells, and also, optionally, substantially free of mature cells. The inventive methods are described in more detail below in the context of two particular exemplary applications: purging of cancer cells from transplants for reinfusion into a patient and cancer diagnostics; and screening for more effective chemotherapeutic agents.

Separating Stem Cell Types, Purging Cancer Cells from Transplants, and Cancer Diagnostics

One application of the inventive methods is the purging of cancer stem/progenitor cells, as well as mature cancer cells, from bone marrow or mobilized peripheral blood transplants for immune system reconstitution. One embodiment of the inventive method for purging cancer cells from the transplants typically involves a two-step cell selection process. In step 1, stem cells and, optionally, progenitor cells are enriched in the initial cell suspension/sample to yield a treated cell suspension enriched in stem cells and, optionally, progenitor cells (including cancer stem and/pr progenitor cells). In step 2, cancer stem/progenitor cells are separated from the non-cancerous stem/progenitor cells to yield a suspension of non-cancerous stem cells that is substantially free of cancer stem cells and cancer progenitor cells.

A variety of cell separation techniques known in the art can potentially be used for forming the enriched stem/progenitor cell suspensions in step 1. Known cell separation methods that can potentially be used in the current invention for forming the enriched stem/progenitor cell suspension include, but are not limited to, antibody-based cell affinity techniques (for one or both of purging non stem/progenitor cells or positively selecting stem/progenitor cells), utilizing, for example, magnetic beads, column affinity chromatography, either of the previous two techniques utilizing avidin-coated substrates and biotinylated antibodies or biotin-coated substrates combined with avidin-coated beads and biotinylated antibodies, fluorescence activated cell sorting (FACS) of various degrees of sophistication (e.g. using a plurality of color channels, low angle and obtuse light scattering detecting channels, impedance channels, etc.), and cell-specific cytotoxin based cell purging, for example using a photosensitive dye that localizes in cells to be purged and destroys them upon activation with light. All of the above mentioned techniques that rely on the specific binding of antibodies to markers on the surface of target cells require that one or more unique marker molecules on the surface of non-cancerous and/or stem cells and cancer stem cells be present and identified. For primitive stem cells and cancer stem cells that may lack such distinguishing cell surface markers or for which such markers may be unknown, purification by affinity based techniques may not be possible. However, for stem cell types(s) having markers that are or can be identified and characterized, affinity based techniques could prove an effective isolation and purging tool. For example, for an embodiment where a marker (or markers) unique to cancer stem cells are or can be identified, the two-step purging strategy outlined above could, alternatively, be performed in a single step, which step comprising the affinity based removal of the mature cancer cells and cancer stem/progenitor cells from the initial sample. Alternatively, for embodiments involving cancer stem cells and mature cancer cells that are or can be found to be lacking a marker (or markers) that is present on the most primitive pluripotent and/or multipotent non-cancerous stem cells, such marker could be used for positively selecting the non-cancerous stem cells substantially free of essentially all cancer cells. However, for embodiments targeting primitive stem cells, which are required for effective reconstitution, and/or primitive cancer stem cells, lacking such known distinguishing cell surface markers, the above one-step affinity methods may not be possible. As will be further discussed herein, it has been observed that many stem cells typically differ in one or more physical properties from more mature cells. For example, typical hematopoietic stem cells have a smaller characteristic size than mature cancer and mature non-cancerous cells. “Characteristic size” or “size” as used herein with respect to dimensions of cells, refers to a linear dimension of a cell from an external surface of the outermost plasma membrane on one side of the geometric center of the cell, through the geometric center of the cell, to an external surface of the outermost plasma membrane on the other side of the geometric center of the cell. For example, for a spherical cell, the characteristic size would be the external diameter of the cell. The typical cancer stem cell or progenitor cell is believed to have a characteristic size and many physical properties that are similar to a corresponding non-cancerous stem cell or progenitor cell respectively. Accordingly, to avoid the limitations of the above-mentioned affinity based methods, certain methods disclosed herein for enriching stem cells can rely, at least in part, on one or more physical differences between stem cells and more mature cells. Specifically, such techniques can be useful in creating a suspension enriched in stem/progenitor cells from the initial precursor cell suspension in step 1 above. While a variety of known methods can potentially be used for physically isolating stem cells in the context of the inventive methods, including, but not limited to filtration and microsieving, flow cytometry and cell sorting based on light scatter differences, centrifugation, and elutriation, two techniques which can have a high degree of selectivity are exemplified below for use in some embodiments of the invention: 1) stem cell enrichment using electric fields; and 2) stem cell enrichment using selective osmotic lysis.

Equipment and conditions for enrichment of stem cells utilizing electric fields is described in commonly owned U.S. Pat. No. 6,043,066, entitled “Cell Separation Using Electric Fields” to Mangano et al., incorporated herein by reference for all purposes, to which the reader is referred for details and guidance. Briefly, the method involves electroporating and inactivating a substantial fraction of cells present in a suspension that have a characteristic electroporation threshold below a predetermined value, for example inactivating a substantial fraction of cells above a certain predetermined threshold size, which threshold value is a function of the electric field and suspension properties, as discussed in greater detail in U.S. Pat. No. 6,043,066, while leaving substantially viable cells having an electroporation threshold above the predetermined value, for example cells below the threshold size.

The term “inactivating” as used herein in reference to the inactivation of cells, for example, mature cells of initial cell suspensions/samples to produce treated suspensions enriched in stem cells and/or progenitor cells, refers to killing and rendering non-viable such cells. In certain embodiments for enriching stem cells and/or progenitor cells, non-stem/progenitor cells may be inactivated and removed, or, alternatively simply removed from the initial suspension. “Removing” cells from a suspension, as used herein in the above context, refers to physical destruction of such cells, for example by lysis, or, alternatively, to physical separation of the cells from the suspension so that the suspension no longer contains the cells.

The term “substantial fraction” as used herein, in the context of inactivating and/or removing cells from a suspension, refers to at least about 25% of such cells being inactivated and/or removed, in some embodiments at least about 50%, in some embodiments at least about 90%, in some embodiments at least about 99%, in some embodiments at least about 99.99999%, and in some embodiments essentially all of the cells being inactivated and/or removed. The term “substantial fraction” has the same meaning as applied in the context of subjecting a suspension of cells to electric field conditions sufficient to porate a “substantial fraction” of a particular type(s) of cells. Such “electric field conditions sufficient to porate” certain cell types in the context of certain embodiments of the invention (e.g. in the context of forming an enriched stem/progenitor cell suspension by exposing a precursor cell suspension to “electric field conditions sufficient to porate” a substantial fraction of non-stem/progenitor cells) can be selected and applied by those of ordinary skill in the art following the teaching of U.S. Pat. No. 6,043,066, previously incorporated by reference. Similarly, “substantially viable” as used herein in the context of describing the viability of cells of a given type in a suspension/sample after a particular treatment, refers to at least about 50% of the cells of the particular type and the population remaining viable, in some embodiments at least about 90%, and in some embodiments at least about 95%.

After employing electric field conditions sufficient to inactivate, and, in some embodiments, irreversibly porate and irreparably lyse, a substantial fraction of cells having a characteristic electroporation threshold which is less than the corresponding threshold for stem cells, gradient density centrifugation and filtration techniques (among other common procedures) can be used to isolate the stem cells from the resulting inactivated cells and/or cellular debris, if desired. “Irreversibly porate,” as used in the context above, refers to poration that is sufficient to cause death and/or physical disruption of the cell without a need for a secondary inactivating step. In some embodiments involving enrichment of stem/progenitor cells utilizing electroporation methods applying electric fields sufficient to porate a substantial fraction of the cells desired to be inactivated but insufficient, alone, to irreversibly porate and inactivate the porated cells that are not stem cells and/or progenitor cells, such porated non-stem/progenitor cells can be selectively inactivated in a secondary inactivating step. For example, in such embodiments, a step can be performed after subjecting the cells suspension to the electric field conditions that includes suspending the cells in a suspension medium different from the suspension medium utilized during the step of subjecting the cells to the electric field conditions. In preferred embodiments, such a second, lysing medium has an ionic strength that is greater than the ionic strength of the medium utilized during the step of subjecting the cells to the electric field conditions.

Purging of mature cancer cells from transplant tissue has been experimentally demonstrated using pulsed electric fields to selectively kill cancerous megakaryocytes and breast cancer cells in suspension with lymphocytes and monocytes (see U.S. Pat. No. 6,043,066 “Examples”—Cases 4 and 5). In preliminary experiments, cancerous megakaryocytes and breast cancer cells were each reduced by a hundred-fold over their initial populations without significant loss of the lymphocyte and monocyte populations. This cell selection was accomplished by selecting the strength of a pulsed electric field in order to efficiently kill the cancer cells by irreversible poration while not adversely impacting the viability of the lymphocyte or monocyte populations. Electric field cell selection techniques can provide over a million-fold reduction in mature cell populations, thus leaving highly enriched stem cell suspensions. (See U.S. Pat. No. 6,043,066 “Examples”—Case 3 for an example of pulsed electric fields being utilized to enrich hematopoietic stem cells.)

Another method for creating an enriched stem and/or progenitor cell suspension in step 1 of the embodiment of inventive method discussed above is via selective colloidal osmotic lysis described in commonly owned copending application Ser. No. 09/690,334 for U.S. patent entitled “Isolation of Hematopoietic Stem Cells and Purging of Tumor Cells From Progenitor Cell Preparations by Means of Osmotic Pressure” to Eppich et al., and corresponding International Application No. PCT/US99/08512, having an International Publication No. WO 99/54439, each incorporated herein by reference, to which the reader is referred for guidance in selecting enrichment conditions. Enrichment of stem cells using osmotic pressure involves utilizing osmotic pressure to selectively lyse the unwanted mature cells. The selection parameter can be the ratio of the nuclear diameter or volume to the cell diameter/volume. In general, the selective osmotic pressure technique for enriching stem and/or progenitor cells and inactivating and/or removing a substantial fraction of the mature cells involves subjecting an initial suspension containing both the stem and/or progenitor cells and mature cells to conditions creating a change in osmotic pressure in the cells so that cells with an average ratio of nuclear volume to total cell volume within a first range of values (i.e. characteristic of mature cell types) are selectively made non-viable relative to cells with an average ratio of nuclear volume to total cell volume within a second range of values (i.e. characteristic of stem cells and/or progenitor cells). In one such embodiment, after employing osmotic pressure to lyse cells having a ratio of nuclear diameter to overall cell diameter which is less than the corresponding ratio for stem cells, gradient density centrifugation and filtration techniques (among other common procedures) may be used to isolate the stem cells from the resulting debris.

Since many different types of stem/progenitor cells are similar to each other in size and ratio of nuclear to cellular volume, step 2 of the exemplary isolation procedure outlined above (namely, separating particular type(s) of stem cells in the stem cell suspension from each other) can be a more difficult task than the depletion of the mature cells in step 1 above. For example, returning to the exemplary embodiments involving cancer stem cells, since many cancer stem cells are believed to be similar in size and ratio of nuclear to cellular volume to non-cancerous stem cells, and, in addition, are believed to have similar or identical expression of cell surface markers, step 2 of the isolation procedure (separating the cancer stem cells in the stem cell suspension from the non-cancerous stem cells) can be a more difficult task than the removal of the mature cells in step 1 above. In the context of hematopoietic reconstitution, the cancer stem cells may be of nearly the same size and morphology as the essential non-cancerous hematopoietic stem cells in the transplant tissue. This may be especially true for the case where the cancer stem cells are transformed hematopoietic stem cells. In some embodiments, the size, morphology, antibody binding and other markers between the cancer and non-cancerous stem cells may be essentially identical, with only the essential genetic material in the nucleus of the cells being different. Such a situation would be especially expected for cancer stem cells corresponding to at least certain leukemias (e.g. CML) and other diseases of the blood and immune system.

In general, the cancer stem cells present in the stem cell suspensions produced by isolating the stem cells in step 1 described above, can, in some embodiments, be epithelial cancer stem cells, as in the case of breast cancer and other epithelial cancers. They can, in some embodiments, also or alternatively be cancer stem cells arising from mutation of the stem cells which produce the mature, differentiated cells for other major organs of the body. They can, in some embodiments, also or alternatively be one of the possible forms of hematopoietic cancer stem cells, with the potential for differentiating into the various cancers of the blood and immune system including the various leukemias.

In general, the inventive techniques described above, and in more detail below, have utility for separating a wide variety of types of cancer stem cells and/or progenitor cells from non-cancerous stem cells and/or progenitor cells and, in some embodiments, for isolating such cancer stem cells and/or progenitor cells to form suspensions that are substantially free of non-cancerous cells. For example, such isolated cancer stem cells and/or progenitor cells can include stem cells for forming cancers that include breast cancer, ovarian cancer, lymphoma, myeloma, leukemia, skin cancer, lung cancer, digestive system cancers, oral cancer, and prostate cancer. The techniques described in more detail below illustrate how cancer stem cells and/or progenitor cells, including those cancer stem cells for forming the various cancers mentioned immediately above, can be isolated and purified and, in some embodiments, characterized and/or used in screening tests for various chemotherapy agents and/or cancer treatment protocols. Also, as described in more detail below, certain embodiments of the inventive methods are able to isolate non-cancerous stem cells and/or progenitor cells from cancer stem cells and/or progenitor cells to form suspensions of the non-cancerous stem cells and/or progenitor cells that are substantially free of cancer cells. As mentioned above, and discussed below, such suspensions can advantageously be utilized, alternatively after expansion and/or differentiation of at least some of the stem cells, for reconstituting the hematopoietic system of human patients in need of a bone marrow transplantation, for example, those undergoing high dose chemotherapy and other marrow-ablating cancer therapies.

In cases involving epithelial cancers, isolating the smallest cells in the bone marrow or mobilized peripheral blood sample from a patient with such a cancer would tend to provide a mix of both the epithelial cancer stem cells and the non-cancerous hematopoietic stem cells. While potentially unlikely, for the reasons discussed above, further separation of these cancer stem cells could potentially be based on size, morphology, antibody binding, or by some other means of differentiation. If separation of cancer stem cells from non-cancerous stem cells can be effected by one or more of the cell separation techniques discussed above, then the selected non-cancerous hematopoietic stem cells can subsequently be expanded using cell culture techniques known in the art (e.g. see Zandstra, A. J., et al. “Advances in hematopoietic stem cell culture,” Curr Opin Biotechnol 9:146-151(1998); Clark B. R. al. “In vitro clonal culture of human hematopoietic progenitor cells,” Methods Mol. Biol. 75: 257-263 (1997); Rice A. et al. “Hematopoietic stem cell expansion,” Transfus. Sci., 18: 263-275 (1997); Eaves C. et al. “Introduction to stem cell biology in vitro. Threshold to the future,” Ann. N.Y. Acad. Sci., 872: 1-8 (1999); Douay L. “Experimental culture conditions are critical for ex vivo expansion of hematopoietic cells,” J. Hematother. Stem Cell Res., 10: 341-346 (2001); Veiby O. P. et al. “Growth factors and hematopoietic stem cells,” Hematol. Oncol. Clin. North Am., 11:1173-1184 (1997); Brugger W. et al. “Ex vivo manipulation of hematopoietic stem and progenitor cells,” Semin. Hematol., 37: 42-49 (2000); McAdams T. A. et al. “Hematopoietic cell culture therapies (Part I): Cell culture considerations.” Trends Biotechnol. 14: 341-349 (1996); Brown et al. “Serum-free culture conditions for cells capable of producing long-term survival in lethally irradiated mice.” Stem Cells, 15:237-245 (1997); Shimakura Y. et al. “Murine stromal cell line HESS-5 maintains reconstituting ability of ex vivo-generated hematopoietic stem cells from human bone marrow and cytokine-mobilized peripheral blood.” Stem Cells, 18: 183-189 (2000); Ahmed N. et al. “Cytokine-induced expansion of human CD34+stem/progenitor and CD34+ CD41+ early megakaryocytic marrow cells cultured on normal osteoblasts.” Stem Cells, 17:92-99 (1999); Zipori D. and Lee F. “Introduction of interleukin-3 gene into stromal cells from the bone marror alters hematopoietic differentiation but does not modify stem cell renewal.” Blood, 71: 586-596 (1988); Simmons P. J. and Haylock D. N. “Use of hematopoietic growth factors for in vitro expansion of precursor cell populations.” Curr Opin. Hematol. 2:189-195 (1995); Scheding S. et al. “Ex vivo expansion of hematopoietic progenitor cells for clinical use.” Semin. Hematol. 35: 232-240 (1998); Hoffman R. “Ex vivo expansion of human hematopoietic stem cells: implications for the modern blood bank.” Vox Sang, 74 Suppl. 2: 259-264 (1998); and U.S. Pat. No. 5,635,386, to Palsson et al. and entitled “Methods for regulating the specific lineages of cells produced in a hematopoietic cell culture;” each of which is incorporated herein by reference) to form the transplant medium for reinfusion into a patient.

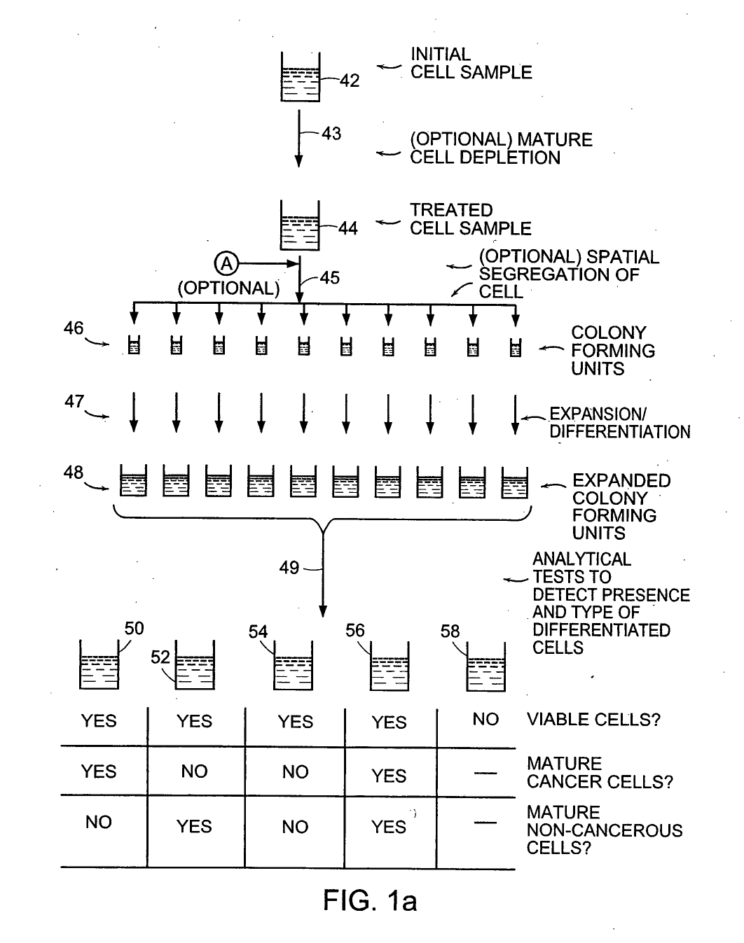

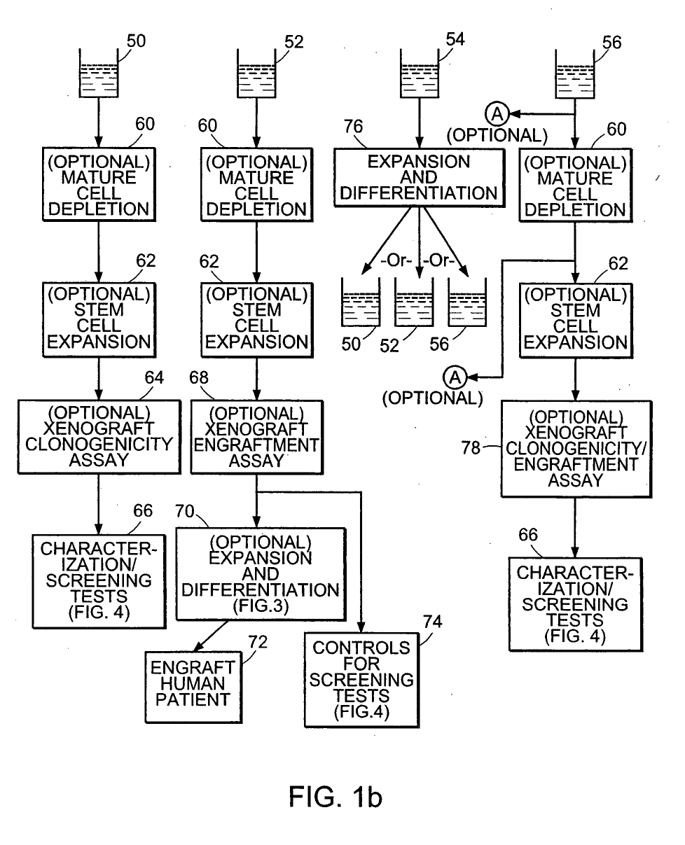

One embodiment for effecting the separation or isolation of cancer stem/progenitor cells from other non-cancerous stem/progenitor cells, which has the flexibility to be utilized with a wide variety of stem cell and tissue types and which can advantageously be utilized when separation of cancer stem/progenitor cells from non-cancerous stem/progenitor by typical prior art cell separation techniques cells is impractical, is shown in FIG. 1a. The inventive cell separation strategy shown in FIG. 1a can be used in certain embodiments to create suspensions containing stem/progenitor cells that are substantially free of all cancer cells (mature, progenitor, and stem), which can then be utilized, after any optional expansion and/or differentiation, as a transplant medium for infusion into a patient. In other embodiments, as further discussed below, the method can be used to create suspensions highly enriched in cancer stem/progenitor cells and substantially free of non-cancerous cells, and, in some embodiments, mature cancer cells. More generally, the method can be employed to remove or isolate any specific type or subpopulation of stem/progenitor cell from sample containing multiple types of stem/progenitor cells.

Referring to FIG. 1a, the illustrated method involves first providing an initial suspension or cell sample 42 of biological cells having a given cell population. In the exemplary embodiment illustrated, initial cell sample 42 will, in certain embodiments, include a mixture of mature cancer cells, mature non-cancerous cells, cancer stem cells and/or progenitor cells, and non-cancerous stem cells and/or progenitor cells. The ultimate goal and final outcome of the illustrative method is the separation of the cancer stem cells and/or progenitor cells from the non-cancerous stem cells and/or progenitor cells. As mentioned above, the initial cell sample 42 may be derived from a wide variety of cell-containing tissues of the body, including, but not limited to, bone marrow, peripheral blood, organs of the body, other solid tissue, solid tumors, etc. In certain embodiments, initial cell sample 42 comprises individual cells in suspension. Tissue samples, for example solid tumors and organs, wherein the cells are not typically arranged as single cell suspensions can be disaggregated, digested, or otherwise processed to yield substantially single cell suspensions of the cell types contained in the tissues by techniques that are well known to those of ordinary skill in the art.

As will be explained in more detail below, one feature of the stem cell separation process of the illustrated method involves partitioning cells contained within and/or derived from the cells of initial cell sample 42 into a plurality of colony forming units for subsequent expansion and differentiation (i.e. in steps 45 and 47 of the illustrated method). In certain embodiments, in order to reduce the number of cells needed to comprise each of the colony forming units in subsequent steps of the method in order to ensure that each unit contains at least a single colony forming stem cell and to reduce contamination of the colony forming units with mature cells, which can potentially complicate and render less accurate the separation and detection of the stem cells within the colony forming units, as described below, an optional step 43 of depleting mature cells present in the precursor cell sample can be performed.

Optional mature cell depletion step 43 can potentially be performed using a variety of cell selection techniques, as described above, which have the ability to selectively inactivate and/or remove from the initial suspension a substantial fraction of the mature cells in the initial suspension, thereby forming a treated suspension enriched in stem cells. In certain embodiments, the depletion technique comprises a technique able to deplete a substantial fraction of, and, in some embodiments, substantially all of, the mature cells of each cell type that is contained in the initial suspension. Two such techniques, which may be able to inactivate and/or remove a substantial fraction of the mature cells of each cell type contained in the initial suspension involve utilizing electric fields or selective osmotic lysis to carry out the optional mature cell depletion of step 43.

When the electric field cell depletion technique discussed above is employed, optional mature cell depletion step 43 comprises subjecting initial suspension 42 to electric field conditions sufficient to porate a substantial fraction of the cells that are not stem cells and/or progenitor cells while maintaining substantially viable the stem cells and/or progenitor cells in the initial cell suspension, and then selectively inactivating a substantial fraction of the porated cells that are not stem cells and/or progenitor cells while maintaining substantially viable the stem cells and/or progenitor cells in the suspension subjected to the electric field conditions.

In some embodiments where the optional mature cell depletion step 43 is performed utilizing selective osmotic lysis, cell depletion can occur by subjecting initial cell suspension 42 to conditions creating a change in osmotic pressure in the cells to that cells with an average ratio of nuclear volume to total cell volume within a first range (i.e. that of the bad characteristic of mature cells) are selectively made non-viable relative to the stem cells and/or progenitor cells which have an average ratio nuclear volume to total cell volume within a second range of value, which is higher than the first range of values.

In alternative embodiments, optional cell depletion step 43 can be effected by removing mature cells from initial cell sample 42 using one or more known techniques such as: affinity cell separation utilizing antibody-coded magnetic beads; affinity cell separation utilizing at least one antibody having specificity to a cell surface marker in combination with avidin-coded beads and biotin-coded columns; microfiltration; florescence activated cell sorting (FACS); cell-specific cited toxic agents; centrifugation; and cell affinity chromatography.

For embodiments of the method illustrated in FIG. 1a wherein an optional mature cell depletion step 43 is performed, a treated cell sample 44 is produced, which has a reduced number of total cells and is enriched in those stem cells and/or progenitor cell that were in initial cell sample 42. In certain embodiments, treated cell sample 44 is substantially free of the mature cells of each cell type that were initially contained in initial cell sample 42.

Steps 45, 47, and 49 comprise the portion of the illustrated method involved in separating the cancer stem cells and/or progenitor cells from the non-cancerous stem cells and/or progenitor cells in treated cell sample 44, or, alternatively, for embodiments wherein optional mature cell depletion step 43 is not performed, in initial cell sample 42. Step 45 evolves spatially segregating the cells of treated cell sample 44 into a plurality of colony forming units 46. Colony forming units 46 are subsequently expanded in step 47 so that the viable stem cells in the units differentiate and increase in number.

A “colony forming unit” as used herein, refers to an individual group of cells that are associated together in spatial proximity (or to a single cell), but that are spatially separated from other cells not in the colony-forming unit, such that upon cell division and propagation of any or all of the cells in a given colony forming unit, a single group of progeny cells can be detected, which group is spatially separated from and able to be differentiated from cells in other colony forming units, or cells derived from cells in other colony forming units.

In certain embodiments, during spatial segregation step 45, cells of treated cell sample 44 are distributed into colony forming units 46 such that each colony forming unit contains only a small number of stem cells or a single stem cell. In certain embodiments, on average, each colony forming unit contains less than about 1,000 stem cells, in certain embodiments less than about 100, in certain embodiments less than about 10, and certain embodiments only a single stem cell. The particular volume of treated cells suspension 44 required to make up each of the colony forming units 46, such that each colony forming unit contains a desired quantity of stem cells, will, of course, depend on the concentration of stem cells contained in treated cell sample 44. In addition, the optimal number of stem cells forming each colony forming unit 46 may depend upon the expected relative concentrations of the different types of stem cells desired to be separated, and/or the particular cell culture conditions chosen for the expansion/differentiation step 47.

Because the concentration and purity of stem cells in treated cell sample 44 (or optionally in precursor cell sample 42) may not always be precisely known or easily determinable, in some embodiments, it may be desirable to prepare colony forming units 46 such that the total number of viable cells included in each colony forming unit is varied, for example by serial dilutions, such that at least some of the colony forming units are expected to include a desired number of colony producing stem cells, as discussed above.

The term “serial dilution” of cells, or any other substance, in a plurality of samples refers simply to the different samples having a different number and/or concentration of the cells or substance therein, such that, for example, a first sample may contain a first number of viable cells and a second sample may contain a second number of viable cells, or a first sample may contain a first concentration of the substance and a second sample may contain second concentration of the substance.

The particular manner in which the spatial segregation of cells occurs in step 45 will depend on the type of cell culture system chosen for the expansion and differentiation step 47. As known to those of ordinary skill in the art, there exist a number of in vitro and in vivo (e.g. xenograft) methods for effecting the culture, expansion, and differentiation of stem cells. A description of a variety of such culture methods can be found, for example, many of the references noted above as describing stem cell expansion and culture techniques, Clarke et al., Bonnet and Dick, Park at al. (incorporated herein by reference), Salmon I and Salmon II (both incorporated herein by reference), and in the following references, each of which is incorporated herein by reference: U.S. Pat. No. 5,750,397 to Tsukamoto et al. entitled “Human hematopoietic stem cell;” Cho R. H. and Muller-Sieburg C. E. “High frequency of long-term culture initiating cells retain in vivo repopulation and self-renewal capacity.” Exp. Hematol., 28: 1080-1086 (2000); Taichman R. S. et al. “Human osteoblasts support human hematopoietic progenitor cells in vitro bone marrow cultures.” Blood, 87: 518-524 (1996) (hereinafter “Taichman et al”); De Wynter E. A. et al. “CD34+AC133+ cells isolated from cord blood are highly enriched in long-term culture-initiating cells, NOD/SCID-repopulating cells and dendritic cell progenitors.” Stem Cells, 16:387-396 (1998); Bruserud O. et al. “New strategies in the treatment of acute myelogenous leukemia (AML): in vitro culture of AML cells—the present use in experimental studies and the possible importance for future therapeutic approaches.” Stem Cells, 19: 1-11 (2001); Greenberger J. S. et al. “Expansion of hematopoietic stem cells in vitro as a model system for human tissue engineering.” Clin. Plast. Surg. 26: 569-578 (1999); Lechner J. F. “Normal human prostrate epithelial cell cultures.” Methods in Cell Biology, 21B: 195-225 (1980); U.S. Pat. No. 5,750,376 to Weiss et al. entitled “In vitro growth and proliferation of genetically modified multipotent neural stem cells and their progeny.”; and U.S. Pat. No. 5,753,506 to Johe entitled “Isolation, propagation and directed differentiation of stem cells from embryonic and adult central nervous system of animals.” Those of ordinary skill in the art, using no more than routine experimentation and optimization, will be able to select or adapt one or more known stem cell expansion/differentiation techniques to for use in culture/expansion and differentiation step 47, which step is described in more detail below.

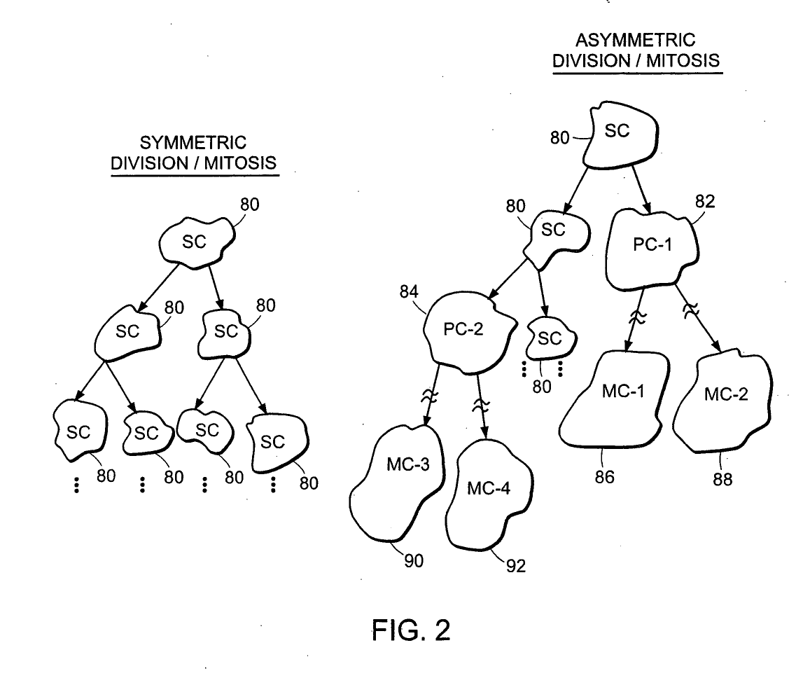

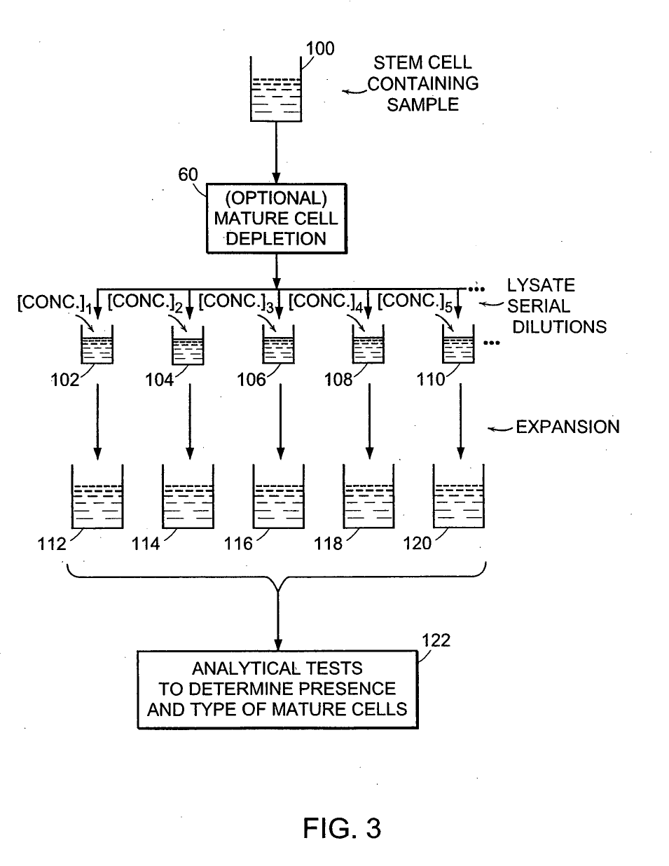

As also will be described in more detail below, in certain embodiments for performing the expansion/differentiation step 47 certain prior art stem cell and cancer stem cell culture techniques can be modified utilizing a novel approach for preferentially inducing stem cells to undergo either symmetric cell division, or asymmetric cell division and differentiation (see below, especially FIGS. 2 and 3 and related discussion).

The particular manner in which spatial segregation of cells occurs in step 45 and the particular characteristics of the of colony forming unit 46 depends upon the type of cell culture system chosen for the colony forming assay culture of step 47. For example, in an embodiment where the expansion and differentiation step 47 is performed in vivo, e.g. in an immunodeficient animal such as a NOD/SCID mouse (see for example, Clarke et al., and Bonnet and Dick), “containers” for containing colony form units 46 would comprise separate immunodeficient animals, for example, separate NOD/SCID mice, and such cells would be spatially segregated by injecting a separate boluses of the treated sample 44 into each immunodeficient animal.

In other embodiments, the formation of the colony forming units 46 in step 45 comprises distributing a predetermined quantity of the cells of treated cell sample 44 into at least one tissue culture container (e.g. a tissue culture plate) containing an appropriate solid or semi-solid tissue culture substrate for culturing the stem cells, such as methylcellulose (see e.g., Bonnet and Dick, and Taichman et al.) or agar (see e.g., Salmon I; Salmon II; and Park et al.). The predetermined quantity of cells to be distributed can be selected so that individual, colony-producing stem cells in the predetermined quantity become spatially separated from each other within the solid or semi-solid tissue culture substrate, each thereby forming one of the plurality of colony forming units 46, such that a plurality of distinguishable colonies forms during the expansion and differentiation step 47.

In certain embodiments, stem cell segregation step 45 involves dividing the treated cell suspension 44 into a plurality of aliquots and placing each of the aliquots into a separate “container”. For embodiment where the cells are expanded and differentiated under in vitro suspension cell culture conditions, the stem cells are cultured freely suspended in the separate container above, which container typically comprises an individual well of a multi-well tissue culture plate, a vial, a tube, a plate, or a flask, etc. Alternatively, for embodiments where colony forming units 46 are expanded in a solid or a semi-solid culture substrate, the separate “container” containing each aliquot, can comprise a well, chamber, a void, or a depression formed in a solid or semi-solid substrate. In each case, each of the aliquots described above comprises one of the colony forming units 46. During step 47, the cells contained in colony forming units 46 are preferably exposed to appropriate cell culture conditions for effecting expansion of the stem cells contained within the colony forming units and also, preferably, differentiation of at least some of the stem cells into progenitor cells and a full variety of the mature cell types normally derived from the stem cells when they are in the body in the tissue compartment for which they are responsible for maintaining.

A variety of cell culture conditions and cell culture media are known and available for propagating stem cells and encouraging self renewal and/or differentiation (see e.g., the references to cell culture techniques referred to and incorporated by reference above). Typically, such formulations utilize a variety of specific exogenous cell growth and differentiation factors (e.g., various interleukins and growth factors) to supplement the cell culture media. Many such known formulations and cell culture techniques are potentially useful for performing the expansion and differentiation step 47 in the context of the present invention, and such media and techniques can be selected and modified, as needed, by those of ordinary skill in the art through routine experimentation and optimization.

In certain embodiments, since the separation of cancer stem cells from non-cancerous stem cells depends upon detection of the identity of mature cell types derived from the stem cells upon expansion and differentiation in step 47 (as described in more detail below on the context of step 49) culture conditions in step 47 are selected to preferentially induce stem cells to propagate by undergoing asymmetric mitosis, whereby each dividing stem cell produces one identical daughter cell and one daughter cell that is more differentiated than the stem cells (see FIG. 2). As explained in more detail below on the context of FIGS. 2 and 3, the present invention provides, in one aspect, a novel stem cell expansion and differentiation technique in which stem cells in culture are exposed to selected concentrations of lysate produced from cells of at least one selected differentiated cell type. As described in more detail below, it is believed that lysate of differentiated and mature cells of types contained in the tissue compartment for which stem cells are responsible for maintaining may contain at least some of the various factors and agents responsible for signaling the stem cell to self renew and/or propagate and/or differentiate along certain lineages. As will be discussed in more detail the context of FIG. 2 below, it is believed that when stem cells are exposed to relatively high concentrations of lysate produced from such mature/differentiated cells, that the stem cells are stimulated to undergo symmetric mitosis, whereby each dividing stem cell produces two identical daughter stem cells. Further, it is believed that lower concentrations of such lysate can induce the stem cells to propagate by preferentially undergoing asymmetric mitosis, leading to terminal differentiation and the production of mature cell types.

Furthermore, it is believed that the identity of the differentiated and/or mature cell from which the lysate is produced can affect the particular lineage(s) of the progeny of the stem cells cultured with the lysate. In other words, and for example, a particular stem cell incubated with lysate derive from one particular mature cell type typically derived from such a stem cell, at a concentration selected to encourage asymmetric division, can preferentially induce such a stem cell to asymmetrically divide such that the progeny of the stem cell differentiate preferentially into the type of mature cell from which the lysate was derived. Accordingly, it is believed that by including cell lysate derived from a mixture of cell types that are characteristic and representative of the cells comprising the tissue compartment for which the cultured stem cell is responsible for maintaining in the body, at a concentration selected to induce asymmetric division and differentiation of the stem cell, that the stem cell can be induced to differentiate and produce the full complement of mature cells typically found in such tissue compartment. The term “tissue compartment,” as used herein, refers to a discrete group of cells in the body, each derived from a particular type of pluripotent, multipotent, or unipotent stem cell, which together form a body structure and/or system for performing a discrete class of functions in the body. Examples include various organs, the hematopoietic system, and various structural tissues, such as the skin, oral tissue, bone, etc.

In those embodiments where cell lysate is utilized in the expansion of differentiation culture of step 47, as mentioned above, it is preferred that such lysate be included in a concentration selected to encourage asymmetric cell division of stem cells in colony forming units 46, thereby leading to the production of mature cell types, which can be detected in expanded colony forming units 48 by the analytical tests described below in more detail in the context of step 49. In order to assure that at least some colony-forming units are cultured with an appropriate concentration of lysate for inducing asymmetric division during expansion, unless it has been predetermined before hand for a particular lysate and stem cell type (e.g., by using the assay described below in the context of FIG. 3), in some embodiments, varying dilutions, e.g. serial dilutions, of the lysate can be included in various colony forming units during the expansion differentiation step 47.

In some embodiments where the inventive method of including cell lysate during stem cell expansion and differentiation is employed, the cell culture media utilized for the expansion, which includes a lysate, can be essentially free of other added differentiation agents and/or growth factors or may include only those factors and agents determined, through routine experimentation and optimization, to be strictly necessary for effecting the expansion and differentiation of the colony forming units 46 so as to form expanded colony forming units, at least some of which contain mature cells derived from stem cells contained in the colony forming units. It should be understood, as would be apparent to those of ordinary skill in the art, that the time allowed for performing the expansion and differentiation culture in step 47 can be selected such that it is long enough to assure that differentiated and mature cells forming the colonies of the expanded colony forming unties are derived from stem cells and not from more differentiated cells that have only limited ability to propagate and differentiate. (see, e.g., Clarke et al., and Bonnet and Dick).

As would also be understood by those skilled in the art, expansion/differentiation step 47 may be repeated two or more times, if desired, to effect a series of serial expansions to further increase the number and/or degree of differentiation of the stem cells in colony forming units 46. In addition, for embodiments wherein more than one step of expansion/differentiation is performed, different cell culture techniques may be utilized for different steps.

Step 49 involves performing analytical tests on the expanded colony forming units 48 to detect the presence of, and determine the type of, mature and/or differentiated cells contained within the expanded colony forming units. A wide variety of cell characterization tests can potentially be used to determine the presence and phenotype of mature and differentiated cells in expanded colony forming units 48, with the particular analytical test chosen being dependent upon the type of cell culture conditions selected in step 47 and/or the type of mature and differentiated cells expected to be derived from the stem cells present in initial cell sample 42. For example, the presence of cells with the expanded colony forming units can be determined by visual observation and/or by bright-field or phase-contrast microscopy. Viability of the cells can be determined through routine dye exclusion test (for example, Trypan blue dye exclusion). These techniques are well known to those of ordinary skill in the art.

A wide variety of known analytical techniques can be utilized to determine the type of mature and/or differentiated as cells present in the expanded colony forming units 48. For example, where expansion and differentiation in step 47 is performed utilizing a colony forming assay culture, wherein the colony forming units are cultured in solid or semi-solid media, mature cancer cells for many types of cancers, can be distinguished from mature non-cancerous cells by visually and/or microscopically observing the morphology of the colony and/or the cells therein. In other embodiments, the type of mature and/or differentiated cells contained within the expanded colony forming units 48 can be determined by immunophenotyping, for example, by incubating at least some of the cells of an expanded colony forming unit with a labeled antibody or other detectable substance having binding specificity for a selected cell surface antigen characteristic of a particular mature and/or differentiated cell type and detecting the label on the cells with an appropriate detection technique, or by employing polymerase chain reaction (PCR) techniques. The above and other analytical tests to characterize the nature of the mature and differentiated cells present in expanded the cell forming units 48 are well known, and those or ordinary skill in the art can readily select an appropriate technique for a specific application.

Upon performing the analytical test(s) to determine the presence of cells and the types of mature and/or differentiated cell in expanding colony forming units 48 in step 49, a variety of categories of expanded colony forming units may be found as indicated at the bottom of FIG. 1a. The types of cell samples comprising expanding colony forming units 48 are likely to fall within five categories characterized by samples 50, 52, 54, 56, and 58, depending upon the number and type of colony-producing stem cells contained within the colony forming unit from which the sample was derived and whether the colony producing stem cell(s) within the colony forming unit underwent symmetric division/mitosis or asymmetric division/mitosis, as discussed above and in more detail below in the context of FIG. 2. Depending upon the particular application and end use desired for the separated stem cells, one or more of samples 50, 52, 54, and 56 may be retained, optionally further processed, and used for various purposes as outlined below in FIG. 1b.

Sample 50 comprises an expanding colony forming unit containing viable cells and mature cancer cells, but being substantially free of mature non-cancerous cells. Sample 50 would be derived from a colony forming unit that a contained cancer stem cell(s) but that may be substantially free of non-cancerous stem cells, and, further, which was induced to undergo asymmetric division during the expansion and differentiation step(s). As shown in FIG. 1b, such a sample may advantageously be used for the characterization of cancer stem cells and for various drug and cancer therapy screening tests.

Sample 52 includes viable cells and mature non-cancerous cells but is substantially free of mature cancer cells. Sample 52 would be derived from a colony forming unit including non-cancerous stem cell(s) and not cancer stem cells, and in which the stem cells within the colony forming unit were induced to undergo asymmetric mitosis. Accordingly, sample 52 would be expected to contain non-cancerous stem cells but to be substantially free of all cancer cells, including cancer stem cells. Such a sample, as discussed below in the context of FIG. 1b, could be advantageously utilized, for embodiments where the sample includes hematopoietic stem cells, for engrafting a human patient to reconstitute the patient's hematopoietic system in the context of a bone marrow transplantation.

Sample 54 contains viable cells, however none of the viable cells are found to display the phenotype of either mature cancer cells or mature non-cancerous cells. Sample 54 would be derived from a colony forming unit including one or both of cancer and non-cancerous stem cells, which colony forming unit was cultured under conditions leading to symmetric division of the stem cells. Such would be the case, for example, for embodiments wherein the colony forming units are incubated with cell lysate as discussed above, wherein the concentration of lysate within the colony forming unit during expansion and differentiation was too high to favor asymmetric division (see discussion) below.

Sample 56 includes viable cells found to include both mature cancer cells and mature non-cancerous cells. Sample 56 would be derived from a colony forming unit in which the colony forming unit included both cancer stem cells and non-cancerous stem cells and which was induced to undergo asymmetric mitosis during the expansion and differentiation step. Sample 56 would be typical of those colony forming units for which the predetermined number of cells included in the colony forming unit was too high such that the colony forming unit included stem cells of both the cancerous and non-cancerous type.

Finally, sample 58 is a sample in which no viable cells were found. Sample 58 would be derived from a colony formed unit that contained no surviving stem cells, indicative, for example, from a colony forming unit in which the initial number of cells added to the unit was too low or in which cell expansion conditions were inappropriate.

FIG. 2 illustrates two of the ways that stem cells, both cancer stem cells and non-cancerous stem cells, can propagate upon expansion. When a stem cell undergoes symmetric division/mitosis as shown in the left of FIG. 2, a stem cell 80 propagates to form two identical daughter stem cells 80, which in turn each propagate to form identical daughter stem cells 80, such that the expanded culture will contain a plurality of stem cells but be relatively deficient in, or substantially free of, more differentiated cells, such as mature cells.

By contrast, stem cells 80 undergoing asymmetric division/mitosis, as shown in the right of FIG. 2, divide to form one identical daughter stem cell 80 and one daughter cell, such as progenitor cell 82, which is more differentiated than the stem cell. Progenitor cells such as progenitor cell 82 and progenitor cell 84 in FIG. 2, in turn differentiate, through one or more generations, to form additional progenitor cells and/or terminally differentiated mature cells 86, 88, 90, and 92.

As discussed above in the context of certain embodiments of expansion/differentiation step 47 of FIG. 1a, it is believed that stem cells can be induced to divide and differentiate differently depending on the extracellular signals received from their surrounding environment. Specifically, as discussed above, without being tied to any particular scientific theory or explanation, it is believed that stem cells are responsive to one or more of a variety of substances, which are released from mature and/or differentiated cells upon cell lysis. It is believed that substances released in such lysate cells can include agents that bind to certain receptors on the stem cell and signal the stem cell to propagate and, under certain conditions, differentiate by asymmetric mitosis.

Furthermore, it is believed that the response of the stem cell to the substances contained in the cell lysate is specific to both the concentration of the lysate and the nature of cells from which the lysate is derived. More specifically, it is believed that stem cells can be induced to differentiate into particular types of cells from which they receive differentiation signaling entities which bind to receptors on the stem cells or otherwise interact with the stem cells. Accordingly, by providing lysate from cells of a specific cell type(s) to a stem cell in culture, which is normally able to differentiate into such a cell type, it is believe that such a stem cell can be induced undergo asymmetric mitosis to produce cells of the particular cell type(s) from which the lysate was derived. Accordingly, if a specific type(s) of mature cell is desired to be derived from stem cells in culture, lysate from such particular cells may be provided to the stem cells in culture to induce them to differentiate to produce expanded numbers of stem cells and one or more of a variety of desired types of differentiated and/or mature cells. Such a cellular cascade would also be expected from at least certain types of progenitor cells. As illustrated above in expansion/differentiation step 47 of FIG. 1a and as discussed in more detail below in the context of FIG. 1b, the inventive culture methods, utilizing cell lysates as described above can be advantageous for performing a variety of the stem cell expansion steps indicated in certain embodiments of the present stem cell separation methods. However, as would be appreciated by those of ordinary skill in the art, the inventive method of inducing stem cells to preferentially undergo symmetric or asymmetric division and to undergo asymmetric division so to produce particular types of mature/differentiated cells, as provided by this aspect of the present invention, also has widespread utility in other applications employing stem cell culture for example for research and/or clinical purposes and can be put to a variety of uses therein, each of which is deemed to be within the scope of the present invention.

As can be appreciated, because it may be difficult to know a priori the specific nature and concentration of the particular factors released upon cell lysis which can induce stem cell expansion and/or differentiation, screening tests and experiments to determine the appropriate concentration of the cell lysate for inducing the desired symmetric or asymmetric division of the stem cells in a stem cell culture may need to be performed to determine the appropriate concentration of lysate for use for a particular purpose. This was discussed above in the context of expansion/differentiation step 47 of FIG. 1a, wherein it was discussed that, in some embodiments, colony forming units 46 can be incubated with variable concentrations (e.g., serial dilutions) of lysate to ensure that the stem cells in at least some of the colony forming units are induced to undergo asymmetric division to produce expanded colony forming units containing differentiated and/or mature cells derived from the stem cells, which can then be detected by the analytical tests of step 49.

FIG. 3 illustrates one general approach to performing such screening tests for determining appropriate concentrations of cell lysate for preferentially inducing symmetric and asymmetric mitosis of stem cell-containing cultures. Such a screening test could be performed within the context of a particular expansion and differentiation step of a stem cell separation method or after- or pre-treatment step thereof (as was the case described above for some embodiments of step 47 of FIG. 1a), or such a screening test can be performed separately, and preferably before such cultures, so as to determine the appropriate concentrations of lysate for performing various expansion steps, without the need to perform variable or serial dilutions at each expansion step.

Referring now to FIG. 3, a screening test for determining an appropriate concentration range of lysate for inducing a desirable type of stem cell expansion can be performed by providing a stem cell-containing sample 100 (e.g., like initial cell sample 42), optionally depleting mature cells from sample 100 in step 60 (which can utilize methods substantially similar to those utilized in step 43 of FIG. 1a), followed by dividing the sample into a plurality of aliquots 102, 104, 106, 108 and 110, which are then cultured and expanded with varying concentrations of cell lysate. Upon expansion, which can utilize various stem cell culture and expansion techniques as described above in the context of step 47 of FIG. 1a, expanded cultures 112, 114, 116, 118 and 120 can be analyzed in step 122 to determine the presence and type of mature cells present in the samples. By determining the presence, type and relative abundance of the mature and/or differentiated cells in the expanded cell cultures, the appropriate concentration range of lysate for preferentially inducing symmetric and asymmetric mitosis of the particular stem cells in the stem cell containing sample 100 can be determined. As previously discussed, while a wide variety of known stem cell culture techniques can be utilized for performing the stem cell expansion, in certain embodiments, so as not to obfuscate the affect of the lysate on the stem cells, it may be preferable to exclude or substantially reduce the amount of exogenous growth factors, interleukins, etc., added to the cultures, except, of course, for those inherently contained within the lysate being tested.

The lysate for use in some of the cell culture embodiments of various methods of the present invention can be produced, by lysing cells using a wide variety of cell lysis techniques well known to those of ordinary skill in the art. Conveniently, for those embodiments of the present method that include an optional mature cell depletion step, such as steps 43 and/or 60, which utilize pulsed electric fields to lyse and deplete mature cells, the lysate resulting from the formation of the mature cell-depleted treated cell sample (e.g., treated cell sample 44) may be utilized, after removal of the viable stem cells remaining in the treated cell sample, as the lysate for use in certain embodiments of the various stem cell expansion steps described herein. If desired, the lysate can be treated before use to remove cell debris and/or to reduce the activity of proteases, nucleases, etc., which may be detrimental to the culture cells.

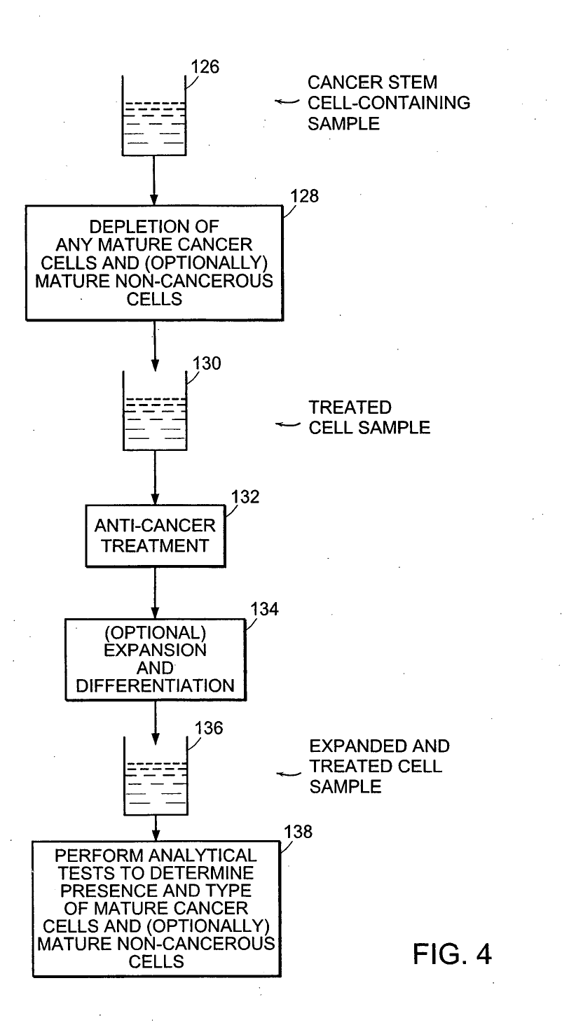

Referring now to FIG. 1b, various potential uses of the purified stem cell-containing samples 52, 54, 56 and 58 described above in the context of FIG. 1a, are illustrated. Sample 50, which is expected to contain cancer stem cells and differentiated/mature cancer cells, but can be substantially free of non-cancerous cells can be utilized, optionally after further processing and expansion steps, for characterization of the cancer stem cells and/or for various cancer drug and treatment protocol screening tests, which are described in more detail below in the context of FIG. 4.

Sample 50 can optionally be subjected to a mature cell depletion step 60 to yield a more highly purified suspension of cancer stem cells (i.e. containing fewer mature cells or, in some embodiments substantially free of mature cells). If greater cell numbers are desired, an optional stem cell expansion step 62 can be performed. In some preferred embodiments, stem cell expansion step 62 would be performed under cell culture conditions including the addition of cell lysate, as described above, at a concentration found to favor symmetric mitosis of the stem cells, thereby producing an expanded cancer stem cell sample with fewer, or substantially no, contaminating mature cells.

In order to confirm that the cancer stem cells contained in sample 50 are true stem cells exhibiting the ability for long-term self renewal and clonogenicity, an optional xenograft clonogenicity/tumorigenicity assay can be performed in step 64. Such assays typically involve injecting a portion of the stem cells contained in the sample into one or more immunodeficient animals and then determining whether the injected cells propagated and differentiated within the animal. Such techniques are known in the art and typically involve immunodeficient mice, especially SCID or NOD/SCID mice (see, e.g., Clarke et al. and Bonnet and Dick). If desired, cancer stem cells can be characterized and further studied in step 66 by utilizing standard characterization techniques, for example those similar to the analytical tests described in the context of step 49 of FIG. 1a. For example, cancer stem cells of a sample could be characterized, as would be apparent to those of ordinary skill in the art, using a variety of techniques including, for example, immunophenotyping based on a variety of known cell surface markers on stem cells, using for example flow cytometry or by employing polymerase chain reaction (PCR) techniques.

Sample 52, which is expected to contain non-cancerous stem cells but which is expected to be substantially free of cancer cells, including cancer stem cells, can be advantageously utilized, for those embodiments wherein the stem cells include lympho-hematopoietic stem cells, after optional further treatment, to engraft a human patient in step 72, and/or as a control for screening tests in step 74 (discussed in more detail below and in the context of FIG. 4). As previously described for sample 50, stem cell containing sample 52 can optionally undergo a mature cell depletion step 60 and, optionally, a further stem cell expansion in step 62, preferably utilizing culture methods including a lysate concentration selected to favor symmetric stem cell division (one or both of these steps are especially useful for embodiments where the stem cells are to be used in screening tests 74).

In addition, for embodiments wherein sample 52 comprises lympho-hematopoietic stem cells, the presence of pluripotent stem cells capable of long-term hematopoietic reconstitution can be assessed utilizing a xenograft engraftment assay in step 68. Such xenograft engraftment assays typically utilize immunodeficient animals, such as SCID mice or NOD/SCID mice, which have been irradiated to ablate their bone marrow cells so that they are in need of hematopoietic reconstitution for survival (see e.g., Clarke et al. and Bonnet and Dick). In some embodiments wherein xenograft engraftment assays 68 and/or xenograft clonogenicity/tumorigenicity assays 64 are performed, in order to further confirm the ability of the stem cells in the sample to undergo long-term self-renewal, a second engraftment on a second immunodeficient animal can be performed by harvesting stem cells derived from the stem cell containing sample and injected into the first immunodeficient animal from the first immunodeficient animal, injecting the harvested cells into the second immunodeficient animal, and determining, in a similar fashion as within the first animal, whether the injected cells propagate and differentiate within the second animal.

For embodiments wherein the stem cells in sample 52 are to be used as controls for screening tests 74, it may be desirable that the stem cells be relatively free of mature cells. Accordingly, for such embodiments, the stem cell sample 52, which can be purified and/or expanded as described above in the context of steps 60 and 62, could be utilized without further processing. However, for embodiments wherein the cells of sample 52 are desired to be used to engraft a human patient for a bone marrow transplant, it may be desirable to perform an optional expansion and differentiation step 70 designed to increase the concentration of stem cells and also induce differentiation via asymmetric mitosis to produce precursor cells and/or mature blood cells of various lineages to ensure more rapid reconstitution of hematopoietic function in the patient. In such embodiments, optional differentiation step 70 can be performed utilizing the inventive cell culture techniques including cell lysate. In preferred embodiments, the lysate would be derived from mature and/or differentiated cells, and more preferably derived from a variety of such cells that are representative and characteristic of the entire range of mature hematopoietic cells. Preferably, the lysate would be included in the culture at a concentration range selected to favor asymmetric division and mitosis. In one embodiment, the lysate is produced by inducing lysis, for example via electric field exposure as described previously, in substantially all of the cells within a sample of bone marrow taken from the patient who is to be engrafted in step 72.

Sample 54, which is expected to contain stem cells of one or both of the cancer and non-cancerous varieties but contain substantially no mature cells may, in some embodiments, simply be discarded. However, in embodiments where the total number of stem cells is at a premium, sample 54 may be further expanded to determine the type(s) of stem cells contained therein. This may be accomplished, for example, by exposing the sample to an expansion and differentiation step 76 under conditions selected to preferentially favor asymmetric division of the stem cells. For example, in embodiments wherein the inventive culture techniques utilizing cell lysate to induce proliferation and differentiation are utilized, sample 54 can be cultured with a concentration of cell lysate within the range found to induce asymmetric stem cell division. Notably, for embodiments wherein sample 54 is derived from a colony forming unit expanded and differentiated in step 47 of FIG. 1a under culture conditions including a concentration of cell lysate, the concentration of lysate selected for the expansion and differentiation step 76 would be selected to be lower than that used in step 47. Otherwise, expansion and differentiation step 76 could be performed identically to step 47. After expansion, one or more analytical tests can be performed, as described for step 49 of the method outlined in FIG. 1a. The resulting expanded samples derived from colony forming units formed from sample 54 should fall into three categories: the first having the characteristics of sample 50 (i.e., including cancer stem cells/mature cells but being substantially free of non-cancerous cells); sample 52 (including non-cancerous stem cells/mature cells but being substantially free of cancer cells); or sample 56 (i.e., including both cancer stem cells and non-cancerous stem cells).

Sample 56 would be expected to include both cancer stem cells and non-cancerous stem cells. Again, as with sample 54, in some embodiments, sample 56 may simply be discarded. Optionally, either before or after an optional mature cell depletion step 60, some or all of sample 56 may be recycled (→A) to spatial segregation step 45 of the method illustrated in FIG. 1a to perform a second attempt at separating the cancer stem cells from the non-cancerous stem cells. Alternatively, because sample 56 is expected to include stem cells of both the cancerous and non-cancerous variety, the sample can be utilized for characterization of either or both stem cell types and/or for performing drug or cancer therapy screening tests 66, as discussed below in FIG. 4. Such samples may be particularly advantageous for performing screening tests, since they would be expected to include both cancerous stem cells and non-cancerous stem cells, thereby including both test and control cells in a single sample. Prior to such uses, sample 56, in addition to optional mature cell depletion step 60, may be further treated by optionally performing a stem cell expansion step 62, preferably under conditions selected to induce symmetric division/mitosis. In addition, and optionally, xenograft clonogenicity/engraftment assay(s), such as those previously described, may be performed to confirm the long-term clonogenicity and/or pluripotency or multipotency of the stem cells in sample 56.

The strategy presented in FIG. 1a can also provide a novel diagnostic tool for detecting a metastatic cancer, determining if a cancer is metastatic, and classifying cancer in a patient both with respect to cancer type and the relative amount of cancer producing cells (e.g. cancer stem cells) in a tissue sample, such as from a solid organ, bone marrow, peripheral blood, or other tissue suspected of having cancer cells. Since micro-metastases are believed to be derived, at least in some cases, from cancer stem cells, the strategy can be especially useful for detecting and classifying such micro-metastases from the circulation and/or tissue remote from sites displaying the primary malignancies. To perform such diagnostic tests, the results of analytical assays 49 to detect mature cancer cells performed on expanded colony-forming units 48 could be used to determine the presence and type of cancer and estimate the relative abundance of cancer stem cells in the test sample 42. For example, if the initial test sample 42 contained 10⁸ cells, and the stem/progenitor cell suspension 44 contained 1000 stem/progenitor cells and was essentially free of mature cancer cells, and each of the colony forming units 46 contained about 10 cells before expansion, if 1 expanded aliquot is found to contain mature cancer cells, then at least 1% of the stem cells in stem/progenitor cell suspension 44 were cancer stem/progenitor cells, and at least 1×10⁻⁵% of the cells in the initial test sample 42 were cancer stem/progenitor cells. The results may then be used to select the most appropriate treatment protocol for the particular patient based on the type and amount of cancer present.

Screening for New, More Effective Drug Candidates/Agents

In the context of anti-cancer therapy, identification of more effective chemotherapeutic agents or cancer treatment protocols in the context of the invention involves, in some embodiments, the development of quantitative tests which can be, in some embodiments, applied quickly and inexpensively in vitro, and which can reliably measure the efficacy of an agent or treatment to be evaluated against mature cancer cells, and cancer stem cells and/or progenitor cells. Such a test can be readily designed using the teachings herein, the level of skill and knowledge possessed by those of ordinary skill in the art, and no more than routine experimentation and/or optimization, to be able to measure not only the efficacy of the agent against the mature cancer cells which make up the bulk of the cancer cells, but also against the primitive cancer stem cells, which may constitute as few as 1 in 10⁶ of the cells present. In the context of the cancer stem cell, a precise definition of what is meant by a more effective chemotherapeutic agent or cancer treatment protocol can be made. In this context, a more effective chemotherapeutic agent or cancer treatment protocol is one which can selectively kill not only mature and differentiating cancer cells, which represent the bulk of the cells present, but also the cancer stem cells themselves, which may be quiescent for extended time periods and therefore may be more difficult to kill. Typical chemotherapeutic agents and cancer treatment protocol available today may not be effective against the cancer stem cell.

Typically, existing in vitro tests perform only the first of these functions (testing efficacy against mature cancer cells), since their efficacy against the cancer stem cell would be masked by the dominating number of dying mature cells present in typical suspensions of cancer cell lines used for testing. To determine the effects of new and existing chemotherapeutic agents and cancer treatment protocol against cancer stem cells, the cancer stem cells can first be isolated from, or enriched with respect to, mature cancer cells, as described above. Then, the chemotherapeutic agents or cancer treatment protocols to be tested can be applied directly to the cancer stem cells, and their effects measured unambiguously, without undo interference from mature cancer cells. The inventive technique can enable a wide spectrum of possibilities arising from combinatorial chemistry and other approaches to new drug discovery to be rapidly and efficiently tested in vitro on cancer stem cells, without necessarily incurring the expense, time, and risks associated with clinical trials in animals and humans.

The present invention, in certain embodiments, provides such a method for screening chemotherapeutic agents or other anti-cancer treatments for efficacy against cancer stem cells and cancer progenitor cells. A flow diagram of one example of such a screening method is shown in FIG. 4. The method involves supplying a suspension 126 (which can, in some embodiments, be similar or identical to samples 50 or 56 described above in the context of FIG. 1b, or such samples as subjected to subsequent treatment steps as outlined in FIG. 1b) that includes mature cancer stem cells. In some embodiments, this suspension could be derived from a specific cultured cancer cell line, or solid tumor type, having essentially only a single type of cancer cell (where “single type” refers to the lineage of the cell but not the state of differentiation). In other embodiments, the suspension can be derived from body tissue, for example, bone marrow or peripheral blood, and may contain non-cancerous cells in addition to the cancer cells (e.g. similar to sample 56 above). The method involves first, essentially eliminating mature cancer cells, if any are present, and, optionally and when present in the initial sample 126, mature non-cancerous cells in step 128 to form a treated stem/progenitor cell suspension 130. The methods employed to perform this stem cell isolation/enrichment step can be the same as those described for steps 43 and 60 of the previously described methods in FIGS. 1a, 1b and 3.

For embodiments utilizing cell separation with electric fields in step 128, the larger, more mature progeny of the cancer stem cells can be killed at lower electric field strengths than the typically smaller cancer stem cells. The strength of the externally applied electric field can be selected, as described above and in more detail in U.S. Pat. No. 6,043,060, previously incorporated by reference, so that the larger differentiating and maturing cells, both cancerous and non-cancerous are irreversibly porated by the applied electric field. The quiescent cancer stem cells, and non-cancerous stem cells when present, would not be irreversibly porated, but would remain substantially viable. The irreversibly porated cells would then die, and the smaller cancer stem cells, and non-cancerous stem cells when present, would survive. Similarly, selective osmotic lysis could potentially be employed to enrich the stem/progenitor cells from the initial cell suspension 126. In this case, the osmotic strength of the suspending medium can be selected, as described in more detail above and in International Application Publication No. WO 99/54439 previously incorporated by reference, to selectively lyse the mature cells, which have a relatively small ratio of nuclear volume to total cell volume, while maintaining the stem/progenitor cells in a substantially viable state.

As an analytical test, suspended cells which survived the stem/progenitor cell selection process can then be tested for potency using the cell culture expansion and/or xenograft assays and analytical techniques discussed previously, if required. Upon expansion, the stem/progenitor cells of the predetermined selected type should, under conditions selected to favor a symmetric division/mitosis described above, produce a spectrum of mature cells expected of the lineage associated with the predetermined selected cell type, which could then be detected by the analytical methods discussed previously. For the example wherein the predetermined selected type is a cancer stem/progenitor cell, upon expansion, the cancer stem/progenitor cells should produce a spectrum of mature cells similar to that expected of the normal cell, with the exception that at least one path leads to mature cancer cells of the type expected, which could then be detected by the analytical methods discussed previously.

In parallel with or subsequent to such analytical tests to verify the presence of the cancer stem cell or other predetermined stem/progenitor cell type, the chemotherapeutic or other drug agents or other cancer treatment protocols could be tested directly against the isolated cancer stem cells and/or non-cancerous stem/progenitor cell type in vitro by treating the stem/progenitor cell suspension 130, which, in the illustrated example contains purified and/or enriched cancer stem cells, with the chemotherapeutic or other drug agent or other cancer treatment protocols to be tested in step 132. The resulting treated suspension can then be cultured and expanded in step 134, as previously described under conditions selected, to increase cell concentration and allow cells to differentiate and mature. The expanded cell suspension 136 can then be subjected to one or more analytical tests 138, using for example any of a variety of known cell phenotyping, viability, functional assays, etc., to determine the effect(s) of such treatment on the viability, growth, differentiation, etc. of the cells of the cultured stem/progenitor cell suspension, for example, to detect the presence and relative amount of cells derived from cancer stem/progenitor cells, e.g. mature cancer cells.

As an example, chemotherapeutic agents or other cancer treatment protocols that are effective against the cancer stem cells should yield expanded cell suspensions 136 that contain relatively few or no detected mature cancer cells. For embodiments where the stem cell enriched suspension 130 included non-cancerous stem cells, treated and expanded suspension 136 could also be subjected to analytical assays to determine the presence and relative quantity of non-cancerous cells. Ideal chemotherapeutic agents or other cancer treatment protocols would be those that yielded expanded suspensions 136 that are substantially free of viable cancer cells but which contain viable non-cancerous cells representing the full complement of cells derivable from the non-cancerous stem cells. Such a result would be indicative of a chemotherapeutic agent or other cancer treatment protocols that is effective against cancer stem cells but which is relatively non-toxic to non-cancerous stem cells. With the inventive screening strategy, combinatorial chemistry and other drug design strategies can be used to rapidly converge on effective chemotherapeutic agents specifically targeted against the cancer stem cell. This procedure can be repeated for all of the various known cancer cell lines so that effective chemotherapeutic agents can be uncovered for a large spectrum of cancer stem cells and cancer progenitor cells.

The function and advantage of these and other embodiments of the present invention may be more fully understood from the examples below. The following examples, while illustrative of certain embodiments of the invention, do not exemplify the full scope of the invention.

### EXAMPLE 1

### Stem Cell Expansion and Differentiation Via an In Vitro Culture Containing Lysate of Differentiated and Mature Cells

The purpose of this example is to show how an appropriate concentration of cell lysate can be determined for culturing lympho-hematopoietic stem cells to induce either symmetric division or asymmetric division.

Peripheral blood mononuclear cells (PBMCs) are obtained by leukopheresis from a cancer patient having chronic myleocytic leukemia (CML). Prior to leukopheresis, the patient is administered granulocyte colony stimulating factor (G-CSF) to mobilize stem cells into the peripheral blood. Leukopheresis is performed such that about 2.5 million CD34+ cells per kilogram body weight is obtained. The resulting cell suspension contains approximately 2×10⁸ leukocytes.

Mature cells are depleted from the sample above by a technique similar to that described in Case 3 of the examples included in U.S. Pat. No. 6,043,066. The pulsed electric field exposure is effective in lysing essentially all of the mature cells in the sample and in enriching CD34+ cells in suspension by at least about a factor 10. The stem cell enriched sample after electric field lysis is then centrifuged, the lysate is collected and saved, and the viable cells are resuspended in appropriate media.

The enriched stem cell suspension is then aliquoted into a plurality of cell culture vessels including an appropriate culture medium for propagating stem cells that is essentially free of exogenous interleukins and other differentiation factors. Serial dilutions of the lysate are made with the aliquots of the enriched stem cell sample. Each of the aliquots are then cultured for a sufficient time to allow the stem cells to propagate and differentiate.

At the termination of cell culture, a portion of each of the expanded aliquots is analyzed by determining total cell number and viability and by observing the cellular morphology of the differentiated cells and/or by immunostaining with fluorescently labeled antibodies specific for cell surface antigens expressed on mature non-cancerous hematopoietic cells (e.g. CD38, CD4, CD8, CD19, CD33, CD3, CD13, CD11B, etc.) and, optionally, markers expressed on differentiated CML cells. As a result of the analytical tests, lysate concentration ranges are determined that result in samples including mature cells (indicative of a symmetric division of stem cells), and samples containing viable cells but few or substantially no detectable cells having a mature phenotype (indicative of a concentration of lysate tending to favor symmetric division of the stem cells).

### EXAMPLE 2

### Cancer Stem Cell Isolation from a Sample Containing Both Cancer Stem Cells and Non-Cancerous Stem Cells

A portion of the stem cell-enriched sample obtained as in Example 1 as a result of performing mature cell depletion via electric field exposure is utilized in the present example to isolate a suspension including cancer stem cells but being substantially free of non-cancerous cells, including non-cancerous stem cells.

The purified stem cell suspension is utilized to prepare a plurality of colony forming assays by plating serial dilutions of the cell suspension in separate culture plates in a semi-solid methylcelluose-containing culture substrate similar to that described in Bonnet and Dick. Cultures are incubated in an appropriate media including a concentration of cell lysate, produced as in Example 1, being present in the culture at a concentration determined in Example 1 to preferentially favor asymmetric mitosis of stem cells.

The cultures are incubated until a plurality of colonies are observed. For each group of plates plated at a particular cell density, the colony is observed and colonies including cells having a leukemic morphology are collected and pooled and the colonies including cells having a morphology of normal hematopoietic cells are collected and pooled in separate containers. The cells in each of these samples are then disaggregated, and the cells are, optionally, further analyzed to determine the presence and type of mature cells, as described above, in Example 1. Samples which are found to include mature leukemic cells but not include mature non-leukemic hematopoietic cells are saved as are samples found to include mature hematopoietic cells and not to include mature leukemic cells (comprising a hematopoietic stem cell-containing sample of substantially free of cancer cells).

The samples including mature leukemic cells and lacking mature non-leukemic cells are then pooled. The resulting suspension comprises leukemic stem cells but is substantially free of non-leukemic stem cells.

### EXAMPLE 3

### Bone Marrow Transplantation of Cancer Stem Cell-Free Hematopoietic Stem Cells

The hematopoietic stem cell-containing sample of Example 2 found to be substantially free of cancer cells is utilized in the present example for reconstituting the bone marrow of the patient described in Example 1 from whom the initial cell sample is derived. It should be understood that the patient, subsequent to cell harvest, will have undergone high dose chemotherapy, radiation, or other marrow-ablating treatment such that the patient is in need of hematopoietic reconstitution.

The sample is expanded in vitro to increase the total number stem cells contained in the sample to prepare for engraftment. At least a portion of the sample undergoing expansion is expanded in culture media containing a concentration of the lysate from Example 1 present within the range found to preferentially favor asymmetric division of the stem cells. Optionally, a portion of the expanded cultures are utilized in a xenograft engraftment assay utilizing lethally irradiated NOD/SCID mice, for example similar to that described in Clarke, et al and Bonnet and Dick. Another portion of the expanded stem cell suspension may be analyzed using, for example, techniques described in Example 1 to confirm the presence of mature hematopoietic cells and/or CFC-GM, BFU-E, CFC-Eo, CFC-GEMM blast forming cells, and immature lymphoid progenitor cells.

The expanded and differentiated stem cell suspension may then be stored until transplantation under suitable conditions, if desired. The patient is then engrafted with at least a portion of the purified stem cell suspension using an appropriate surgical technique known to those skilled in the art. The patient is subsequently found to enjoy complete hematopoietic reconstitution and indefinite remission of leukemia.

### EXAMPLE 4

### Chemotherapeutic Drug Screening with Purified Cancer Stem Cells

In this example, the isolated stem cell sample produced according to Example 2 that was found to contain cancer stem cells but was found to be substantially free of mature non-cancerous cells is utilized to screen the effectiveness of chemotherapeutic agents against cancer stem cells.

The cancer stem cell containing sample is first subjected to electric field conditions selected to inactivate the mature cancer cells present in the sample while leaving substantially viable the cancer stem cells. The conditions utilized are substantially similar to those described above in Example 1. This treated cell sample containing purified cancer stem cells is then exposed to the chemotherapeutic agents in concentrations and under conditions significant with respect to the proposed use of these compounds in vivo. The treated sample is then expanded in vitro with cell lysate as produced in Example 1 included in the culture at a concentration selected to favor asymmetric mitosis under conditions as previously described in the above examples. The expanded cell sample is then subjected to analytical tests, as described in the above examples, to determine the presence and type of any cells that happen to be present. In parallel, a sample of purified stem cells including non-cancerous hematopoietic stem cells but excluding cancer stem cells, as produced in Example 3 is subjected to identical treatment as the cancer stem cell containing sample above. After the expansion and differentiation step, the expanded control sample comprising non-cancer stem cells and being free of cancer stem cells is also subjected to analytical tests to determine the presence and type of any mature cells present. Desirably, the test agent should result in the expanded cancer stem cell-containing sample including few or substantially no viable cells, while the sample containing non-cancerous stem cells, upon expansion, should be found to include mature hematopoietic cells and/or non-cancerous blast forming progenitor cells.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, configurations, etc described herein are meant to be exemplary and that actual parameters, dimensions, materials, configurations, etc. will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material, composition, and/or method described herein. In addition, any combination of two or more such features, systems, materials, compositions, and/or methods, provided that such features, systems, materials, compositions, and/or methods are not mutually inconsistent, is included within the scope of the present invention. In the claims (as well as in the specification above), all transitional phrases or phrases of inclusion, such as “comprising,” “including,” “carrying,” “having,” “containing,” “composed of,” “made of,” “formed of” and the like shall be interpreted to be open-ended, i.e. to mean “including but not limited to.” Only the transitional phrases or phrases of inclusion “consisting of” and “consisting essentially of” are to be interpreted as closed or semi-closed phrases, respectively, as set forth in MPEP section 2111.03.

What is claimed:

1. A method for obtaining a cancer stem cell from a mixture of cell types comprising:
a. from an initial suspension of biological cells, the cells having a given cell population including mature cancer cells, mature non-cancerous cells, cancer stem cells, and non-cancerous stem cells, forming a treated cell suspension by enriching the initial suspension in the cancer stem cells by inactivating and/or removing from the initial suspension a substantial fraction of the mature cells of each cell type contained in the initial suspension;
b. spatially segregating the cells of the treated cell suspension into a plurality of colony forming units;
c. expanding the colony forming units so that the viable cancer stem cells in the colony forming units differentiate and increase in number;
d. performing at least one analytical test on at least one of the expanded colony forming units of step (c) to detect the presence of mature cancer cells; and
e. retaining the cells of at least one of the expanded colony-forming units having a desired cell type as determined by the at least one analytical test performed in step (d).

2. The method as in claim 1, wherein step (b) comprises:
distributing a predetermined quantity of the cells of the treated suspension from step (a) into at least one tissue culture container containing an appropriate solid or semi-solid tissue culture substrate for the cells to be cultured.

3. The method as in claim 2, wherein the predetermined quantity of cells distributed in the distributing step is selected so that individual, colony-forming stem cells in the predetermined quantity become spatially separated from each other, each thereby forming one of the plurality of colony forming units, such that the stem cells form a plurality of individually distinguishable colonies during step (c).

4. The method as in claim 1, wherein step (b) comprises:
dividing the treated cell suspension into a plurality of aliquots, each aliquot having a volume selected to contain at least one viable cell.

5. The method as in claim 4, wherein the volume of at least one aliquot is selected to contain, on average, a single colony-forming stem cell.

6. The method as in claim 5, wherein the dividing step comprises preparing a series of dilutions of the cells in the treated cell suspension and forming the plurality of aliquots, from the dilutions such that a first aliquot contains a first number of viable cells and a second aliquot contains a second number of viable cells.

7. The method as in claim 4, wherein step (b) comprises:
placing each of the aliquots into a separate container, each aliquot comprising a separate colony-forming unit.

8. The method as in claim 7, wherein in step (c), the colony-forming units are expanded under in vitro suspension cell culture conditions, and wherein the separate container comprises an individual well of a multi-well tissue culture plate, a vial, a tube, a plate, or a flask.

9. The method as in claim 7, wherein in step (c), the colony-forming units are expanded in a solid or semi-solid culture substrate, and wherein the separate container comprises a well, a chamber, a void, or a depression formed in the solid or semi-solid substrate.

10. The method as in claim 1, wherein step (d) comprises visually and/or microscopically observing the morphology of the cells of the at least one expanded colony-forming units.

11. The method as in claim 1, wherein step (d) comprises incubating at least some of the cells of the at least one expanded colony-forming unit with a labeled antibody having binding specificity for a selected cell surface antigen characteristic of a particular mature cell type.

12. The method as in claim 1, further comprising after step (e):
inactivating and/or removing from at least a portion of the at least one expanded colony-forming unit a substantial fraction of any mature cells contained in the at least a portion of the expanded colony-forming unit.

13. The method as in claim 1, further comprising after step (e):
further expanding at least a portion of the at least one expanded colony forming unit so that the viable cancer stem cells in the at least a portion of the colony forming unit increases in number.

14. The method as in claim 13, wherein the at least a portion of the at least one expanded colony forming unit is further expanded via in vitro cell culture methods.

15. The method as in claim 1, wherein the cells of the at least one expanded colony-forming unit retained in step (e) include cancer stem cells but are substantially free of non-cancerous cells.

16. The method as in claim 15, further comprising:
testing the clonogenicity of the cells of the at least one expanded colony-forming unit retained in step (e) by injecting at least some of the cells into a first immunodeficient animal and determining whether the injected cells propagate and differentiate within the animal.

17. The method as in claim 16, wherein the immunodeficient animal comprises a NOD/SCID mouse.

18. The method as in claim 16, wherein in the testing step, long-term clonogenicity of the cells of the at least one expanded colony-forming unit retained in step (e) is determined by performing a second engraftment on a second immunodeficient animal by harvesting cancer stem cells derived from the at least one expanded colony-forming unit injected into the first immunodeficient animal from the first immunodeficient animal, injecting the harvested cells into the second immunodeficient animal, and determining whether the injected cells propagate and differentiate within the animal.

19. The method as in claim 15, further comprising:
screening at least one of a chemotherapeutic agent and/or cancer treatment protocol for efficacy against the cancer stem cells included in the at least one expanded colony-forming unit retained in step (e).

20. A method for obtaining a desired type of stem and/or progenitor cell from a mixture of cell types comprising:
a. forming a treated suspension by enriching an initial suspension of biological cells, having a given cell population including mature cancer cells, mature non-cancerous cells, cancer stem cells and/or progenitor cells, and non-cancerous stem cells and/or progenitor cells, in the cancer stem cells and/or progenitor cells and the non-cancerous stem cells and/or progenitor cells by inactivating and/or removing from the initial suspension a substantial fraction of the mature cells of each cell type contained in the initial suspension; and
b. separating the cancer stem cells and/or progenitor cells from the non-cancerous stem cells and/or progenitor cells.

21. A method for obtaining a desired type of stem and/or progenitor cell from a mixture of cell types comprising:
a. subjecting an initial suspension of biological cells, having a given cell population including mature cancer cells, mature non-cancerous cells, cancer stem cells and/or progenitor cells, and non-cancerous stem cells and/or progenitor cells, to electric field conditions sufficient to porate a substantial fraction of cells that are not stem cells and/or progenitor cells while maintaining substantially viable the stem cells and/or precursor cells in the initial suspension;
b. selectively inactivating a substantial fraction of the porated cells that are not stem cells and/or progenitor cells while maintaining substantially viable the stem cells and/or progenitor cells in the suspension subjected to the electric field conditions in step (a); and
c. separating the cancer stem cells and/or progenitor cells from the non-cancerous stem cells and/or progenitor cells.
